SEQUENCE LISTING (9 sequences)

SEQ ID NO: 1 - DNA, 2204 bp, Homo sapiens
gtggggccccagagcgacgctgagtgcgtgcgggactcggagtacgtgacggagccccgagctctcatgcccgccacgccgccccgggccatcccccggagccccggctccgcacaccccagttcggctcaccggtcctatctggggccagagtttcgcccgcaccactacagggccgctggggagtcggggccccccagatctgcccgcctcaagtccgcgggacgtcacccccctttccacgctactgcagccgtcgcagtcccacccctttccgggaggtgagggaatgagtgacggctctcccgacgaatggcgaggcggagctgagggggcgtgccccggaggcgggaagtgggtggggctcgccttagctaggcaggaagtcggcgcgggcggcgcggacagtatctgtgggtacccggagcacggagatctcgccggctttacgttcacctcggtgtctgcagcaccctccgcttcctctcctaggcgacgagacccagtggctagaagttcaccatgtctattctcaagatccatgccagggagatctttgactctcgcgggaatcccactgttgaggttgatctcttcacctcaaaaggtctcttcagagctgctgtgcccagtggtgcttcaactggtatctatgaggccctagagctccgggacaatgataagactcgctatatggggaagggtgtctcaaaggctgttgagcacatcaataaaactattgcgcctgccctggttagcaagaaactgaacgtcacagaacaagagaagattgacaaactgatgatcgagatggatggaacagaaaataaatctaagtttggtgcgaacgccattctgggggtgtcccttgccgtctgcaaagctggtgccgttgagaagggggtccccctgtaccgccacatcgctgacttggctggcaactctgaagtcatcctgccagtcccggcgttcaatgtcatcaatggcggttctcatgctggcaacaagctggccatgcaggagttcatgatcctcccagtcggtgcagcaaacttcagggaagccatgcgcattggagcagaggtttaccacaacctgaagaatgtcatcaaggagaaatatgggaaagatgccaccaatgtgggggatgaaggcgggtttgctcccaacatcctggagaataaagaaggcctggagctgctgaagactgctattgggaaagctggctacactgataaggtggtcatcggcatggacgtagcggcctccgagttcttcaggtctgggaagtatgacctggacttcaagtctcccgatgaccccagcaggtacatctcgcctgaccagctggctgacctgtacaagtccttcatcaaggactacccagtggtgtctatcgaagatccctttgaccaggatgactggggagcttggcagaagttcacagccagtgcaggaatccaggtagtgggggatgatctcacagtgaccaacccaaagaggatcgccaaggccgtgaacgagaagtcctgcaactgcctcctgctcaaagtcaaccagattggctccgtgaccgagtctcttcaggcgtgcaagctggcccaggccaatggttggggcgtcatggtgtctcatcgttcgggggagactgaagataccttcatcgctgacctggttgtggggctgtgcactgggcagatcaagactggtgccccttgccgatctgagcgcttggccaagtacaaccagctcctcagaattgaagaggagctgggcagcaaggctaagtttgccggcaggaacttcagaaaccccttggccaagtaagctgtgggcaggcaagcccttcggtcacctgttggctacacagacccctcccctcgtgtcagctcaggcagctcgaggcccccgaccaacacttgcaggggtccctgctagttagcgccccaccgccgtggagttcgtaccgcttccttagaacttctacagaagccaagctccctggagccctgttggcagctctagctttgcagtcgtgtaattggcccaagtcattgtttttctcgcctcactttccaccaagtgtctagagtcatgtgagcctcgtgtcatctccggggtggccacaggctagatccccggtggttttgtgctcaaaataaaaagcctcagtgacccatgagaataaaaaaaaaaaaaaaaaa

SEQ ID NO: 2 - protein, 434 aa, Homo sapiens
MSILKIHAREIFDSRGNPTVEVDLFTSKGLFRAAVPSGASTGIYEALELRDNDKTRYMGKGVSKAVEHINKTIAPALVSKKLNVTEQEKIDKLMIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 3 - DNA, 2567 bp, Homo sapiens
aactaaagaaaagtttccccatctcccaggagggttctgtgggccctccagagatcatcagcctcttcacgggctagaaaggatccagggaaggtctaaccaatgacctgccctgaatggtgagctgcaggtgtgtcatttagtgtgattttcctgttgactgactcataggagccctgctctgtggcagagctagcctctggctgtattcaaattgacttagtgtgtgtgcaacattgacctttctagagatagaacatgtggccaaattacagaaaagcacatagggctagatcacgcattctcagtggggcacccggaaaactccaaaaaggctgcagggaggggacaatgatgaaatcaggttgtgaaacactgggctggtgtcgcagtggtggtgctgggtgttcagtcccgctttaatgctgtaagaagcactctacacacacgaacatgttaccatttgaccgttgtttaatggcgtacgtggggacttagccggagcaggatgatgctgtgccttgatggtaatgagtgctcagtaagtaagcatttgtggaagattgaacgcatggcccctgaaatgctctcctctgctttcctgccccctcactgtctctcactcgcagtccttaatcaccggttctcttctgagtctctctcatttttccttcttcatcctctgctgggcaggcgtctccagacccattaagtatattaatgagttcctggcaccagccctgtgcactcaggtaactgattgaacagcctttagtctgcagttggcgtttccagtgcatggtcttgcaaactaacctccagtcagatcgttctgagccagctgctgttttgtgtggctctaaccctctggggtcctaggtaggagcactcagactgggccggaaagtcctccgattctggggggaaaggggagagggggaagaggtcccacagaaggtcccttggtgggcttccgcgtcggcctcaacagtggttctctctaacaatgctgctcaagcctgttttaaagttaatgtcagtaatttgatttgattgttccttccaggtgtctcaaaggctgttgagcacatcaataaaactattgcgcctgccctggttagcaagaaactgaacgtcacagaacaagagaagattgacaaactgatgatcgagatggatggaacagaaaataaatctaagtttggtgcgaacgccattctgggggtgtcccttgccgtctgcaaagctggtgccgttgagaagggggtccccctgtaccgccacatcgctgacttggctggcaactctgaagtcatcctgccagtcccggcgttcaatgtcatcaatggcggttctcatgctggcaacaagctggccatgcaggagttcatgatcctcccagtcggtgcagcaaacttcagggaagccatgcgcattggagcagaggtttaccacaacctgaagaatgtcatcaaggagaaatatgggaaagatgccaccaatgtgggggatgaaggcgggtttgctcccaacatcctggagaataaagaaggcctggagctgctgaagactgctattgggaaagctggctacactgataaggtggtcatcggcatggacgtagcggcctccgagttcttcaggtctgggaagtatgacctggacttcaagtctcccgatgaccccagcaggtacatctcgcctgaccagctggctgacctgtacaagtccttcatcaaggactacccagtggtgtctatcgaagatccctttgaccaggatgactggggagcttggcagaagttcacagccagtgcaggaatccaggtagtgggggatgatctcacagtgaccaacccaaagaggatcgccaaggccgtgaacgagaagtcctgcaactgcctcctgctcaaagtcaaccagattggctccgtgaccgagtctcttcaggcgtgcaagctggcccaggccaatggttggggcgtcatggtgtctcatcgttcgggggagactgaagataccttcatcgctgacctggttgtggggctgtgcactgggcagatcaagactggtgccccttgccgatctgagcgcttggccaagtacaaccagctcctcagaattgaagaggagctgggcagcaaggctaagtttgccggcaggaacttcagaaaccccttggccaagtaagctgtgggcaggcaagcccttcggtcacctgttggctacacagacccctcccctcgtgtcagctcaggcagctcgaggcccccgaccaacacttgcaggggtccctgctagttagcgccccaccgccgtggagttcgtaccgcttccttagaacttctacagaagccaagctccctggagccctgttggcagctctagctttgcagtcgtgtaattggcccaagtcattgtttttctcgcctcactttccaccaagtgtctagagtcatgtgagcctcgtgtcatctccggggtggccacaggctagatccccggtggttttgtgctcaaaataaaaagcctcagtgacccatgagaataaaaaaaaaaaaaaaaaa

SEQ ID NO: 4 - protein, 341 aa, Homo sapiens
MIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 5 - protein, 7 aa, Artificial Sequence
  muscle targeting peptide
ASSLNIA

SEQ ID NO: 6 - protein, 7 aa, Artificial Sequence
  muscle targeting peptide
WDANGKT

SEQ ID NO: 7 - protein, 7 aa, Artificial Sequence
  muscle targeting peptide
GETRAPL

SEQ ID NO: 8 - protein, 9 aa, Artificial Sequence
  muscle targeting peptide
CGHHPVYAC

SEQ ID NO: 9 - protein, 7 aa, Artificial Sequence
  muscle targeting peptide
HAIYPRH

(19) United States
(12) Patent Application Publication
(10) Pub. No.: US 2016/0375113 A1
(43) Pub. Date: Dec. 29, 2016

(54) COMPOSITIONS COMPRISING ENO1 AND THEIR USE IN METHODS OF TREATING OBESITY OR OVERWEIGHT AND REDUCING WEIGHT GAIN

(71) Applicant: Berg LLC, Nashville, TN (US)

(72) Inventors: Niven Rajin Narain, Cambridge, MA (US); Rangaprasad Sarangarajan, Boylston, MA (US); Vivek K. Vishnudas, Bedford, MA (US); Stephane Gesta, Arlington, MA (US); Enxuan Jing, West Roxbury, MA (US)

(21) Appl. No.: 15/190,126

(22) Filed: Jun. 22, 2016

Related U.S. Application Data
(60) Provisional application No. 62/182,966, filed on Jun. 22, 2015.

(51) Int. Cl. A61K 38/51; A61K 9/00

(52) U.S. Cl. CPC A61K 38/51; C12Y 402/01011; A61K 9/0053; A61K 9/0019

(57) ABSTRACT

The invention provides a method for reducing or preventing body weight gain in a subject comprising administering to the subject enolase 1 (Eno1). The invention also provides methods of treating obesity, and of reducing body weight in a subject afflicted with an overweight condition, comprising administering to the subject enolase 1 (Eno1). In certain embodiments, the body weight gain, obesity or overweight condition is caused by a therapeutic treatment, such as a diabetic drug. In certain methods of the invention, the Eno1 is delivered to muscle.

### RELATED APPLICATIONS

This application claims priority to U.S. Provisional Patent Application No. 62/182,966 filed on Jun. 22, 2015, the contents of which are incorporated herein in their entirety.

### SUBMISSION OF SEQUENCE LISTING

The Sequence Listing associated with this application is filed in electronic format via EFS-Web and hereby incorporated by reference into the specification in its entirety. The name of the text file containing the Sequence Listing is 119992_14802_Sequence_Listing. The size of the text file is 15 KB, and the text file was created on Jun. 22, 2016.

### BACKGROUND

Obesity is a major public health problem in developed countries. It is also increasing steadily in developing countries and is affecting an ever younger population. For example, in the United States, obesity affects over 20% of men and over 25% of women. Patients having a body mass index (BMI=weight (kg)/height² (m²)) greater than or equal to 30 are considered to be obese (Int. J. Obes., 1998, 22, 39-47; Obesity Lancet, 1997, 350, 423-426).

Obesity is a chronic disorder of energy imbalance characterized by an excess of energy intake in the long term compared with limited energy expenditure, leading to storage of the excess energy in the form of white adipose tissue. Excess adipose tissue directly contributes to problems of fatigue, shortness of breath, sleep apnea and osteoarthritis (see US 2006/0002911).

Obesity and overweight can have various origins: they may come about following deregulation of food intake, following hormonal disturbance, or following administration of a therapeutic treatment. For example, treating type II diabetes with rosiglitazone (Avandia) or sulphonylureas causes patients to gain weight. Similarly, in type I (insulin-dependent) diabetes, insulin therapy is also a cause of weight gain in patients (In Progress in Obesity Research, 8th International Congress on Obesity, 1999, 739-746; Annals of Internal Medicine, 1998, 128, 165-175).

Furthermore, obesity is a well-established risk factor for the development of insulin resistance, of dyslipidaemia and, ultimately, of non-insulin-dependent diabetes. It is a factor contributing to cardiovascular diseases and is associated with a significantly increased risk of cerebro-vascular accidents, vesicular calculi, respiratory dysfunction, osteoarthritis, several forms of cancer and premature death.

Thus a need exists for therapeutic agents for the treatment and prevention of obesity, overweight and body weight gain.

### SUMMARY OF THE INVENTION

In one aspect, the invention provides a method of treating obesity in a subject in need thereof, comprising administering to the subject a therapeutically effective amount of a composition comprising Eno1 or a fragment thereof, thereby treating obesity in the subject. In certain embodiments, the subject is suffering from obesity, and the obesity is type 2 diabetes, type 1 diabetes, or pre-diabetes. In certain embodiments, the obesity is caused by a therapeutic treatment. In certain embodiments, the therapeutic treatment is a diabetic drug.

In one aspect, the invention provides a method of reducing body weight in a subject afflicted with an overweight condition, comprising administering to the subject a therapeutically effective amount of a composition comprising Eno1 or a fragment thereof, thereby reducing body weight in the subject. In certain embodiments, the subject has a body mass index of between 25 kg/m² and 30 kg/m². In certain embodiments, the overweight condition is caused by a therapeutic treatment. In certain embodiments, the therapeutic treatment is a diabetic drug.

In one aspect, the invention provides a method of reducing or preventing body weight gain in a subject, comprising administering to the subject a therapeutically effective amount of a composition comprising Eno1 or a fragment thereof, thereby reducing or preventing body weight gain in the subject. In certain embodiments, the subject is in need of a therapeutic treatment that induces weight gain. In certain embodiments, the subject is undergoing a therapeutic treatment that induces weight gain. In certain embodiments, the therapeutic treatment is a diabetic drug. In certain embodiments, the diabetic drug is selected from the group consisting of sulfonylureas, insulin, GLP-1 receptor agonists, DPP-4 inhibitors, metformin, and rosiglitazone. In certain embodiments, the diabetic drug is rosiglitazone. In certain embodiments, the subject is afflicted with diabetes. In certain embodiments, the diabetes is type 2 diabetes, type 1 diabetes, or pre-diabetes.

In certain embodiments of the aforementioned methods, administering Eno1 to the subject reduces body weight by at least 5% relative to a control. In certain embodiments, administering Eno1 to the subject reduces body mass index (BMI) by at least 5% relative to a control. In certain embodiments, the subject has any one or more of elevated blood glucose, decreased glucose tolerance, decreased insulin sensitivity and/or insulin resistance, diabetes, elevated Hb1Ac level, and abnormal blood glucose level control. In certain embodiments, the method further comprises selecting a subject having any one or more of obesity, elevated blood glucose, decreased glucose tolerance, decreased insulin sensitivity and/or insulin resistance, diabetes, elevated Hb1Ac level, and abnormal blood glucose level control. In certain embodiments, the subject is human. In certain embodiments, the Eno1 or fragment thereof comprises an Eno1 polypeptide or a fragment thereof. In certain embodiments, the Eno1 or fragment thereof comprises an Eno1 nucleic acid or a fragment thereof. In certain embodiments, the Eno1 nucleic acid or fragment thereof is present in an expression vector. In certain embodiments, the Eno1 or fragment thereof is biologically active. In certain embodiments, the Eno1 or fragment thereof has at least 90% activity of a purified endogenous human Eno1 polypeptide. In certain embodiments, the Eno1 is human Eno1.

In certain embodiments of the aforementioned methods, the composition comprising Eno1 or a fragment thereof is for delivery to a muscle cell. In certain embodiments, the composition further comprises a muscle targeting moiety. In certain embodiments, the muscle targeting moiety is a muscle targeting peptide. In certain embodiments, the Eno1 polypeptide or fragment thereof and the muscle targeting peptide are present in a complex. In certain embodiments, the muscle targeting peptide comprises an amino acid sequence selected from the group consisting of: ASSLNIA (SEQ ID NO: 7); WDANGKT (SEQ ID NO: 8); GETRAPL (SEQ ID NO: 9); CGHHPVYAC (SEQ ID NO: 5); and HAIYPRH (SEQ ID NO: 6). In certain embodiments, the complex further comprises a linker. In certain embodiments, the linker is selected from the group consisting of a covalent linker, a non-covalent linkage, and a reversible linker. In certain embodiments, the linker comprises a protease cleavage site. In certain embodiments, the Eno1 is released from the complex upon delivery to a muscle cell. In certain embodiments, the Eno1 and the muscle targeting peptide are present in the complex at a ratio of about 1:1 to about 1:30. In certain embodiments, the composition further comprises a liposome. In certain embodiments, the Eno1 is administered orally. In certain embodiments, the Eno1 is administered parenterally. In certain embodiments, the Eno1 is administered by a route selected from the group consisting of intramuscular, intravenous, and subcutaneous.

### BRIEF DESCRIPTION OF THE DRAWINGS

FIG. 1 shows the effect of rosiglitazone and Eno1 on body weight in a diabetic mouse model (db/db mice). Treatment groups shown are Saline_Lean (saline treatment of lean mice); Saline-db (saline treatment of db/db mice); Rosi (rosiglitazone treatment of db/db mice, 20 mg/kg/day); and Rosi+Eno1 (combination of 20 mg/kg/day rosiglitazone and 400 μg/kg/day Eno1 treatment of db/db mice). Rosiglitazone alone and rosiglitazone+Eno1 showed increased body weight compared to control (saline treated) db/db mice. However, body weight was lower in the rosiglitazone+Eno1 treatment group compared to rosiglitazone alone, indicating that Eno1 attenuates rosiglitazone induced weight gain.

FIG. 2 shows the effect of rosiglitazone and Eno1 on gained body weight in a diabetic mouse model (db/db mice). Treatment groups shown are Saline_Lean (saline treatment of lean mice); Saline-db (saline treatment of db/db mice); Rosi (rosiglitazone treatment of db/db mice, 20 mg/kg/day); and Rosi+Eno1 (combination of 20 mg/kg/day rosiglitazone and 400 μg/kg/day Eno1 treatment of db/db mice). Diabetic mice treated with rosiglitazone alone or rosiglitazone+Eno1 gained more body weight than control (saline treated) db/db mice. Body weight gain in Rosiglitazone treated mice was attenuated when mice were also administered Eno1.

FIG. 3 shows the effect of rosiglitazone and Eno1 on fed blood glucose levels in a diabetic mouse model (db/db mice). Treatment groups shown are Saline_Lean (saline treatment of lean mice); Saline-db (saline treatment of db/db mice); Rosi (rosiglitazone treatment of db/db mice, 20 mg/kg/day); and Rosi+Eno1 (combination of 20 mg/kg/day rosiglitazone and 400 μg/kg/day Eno1 treatment of db/db mice). The combination of rosiglitazone and Eno1 reduced blood glucose levels more quickly than rosiglitazone alone.

FIGS. 4A and 4B show the (A) amino acid (SEQ ID NO: 2) and (B) nucleic acid coding sequence (SEQ ID NO: 1) of human Eno1, variant 1 (NCBI Accession No. NM_001428.3).

FIGS. 5A and 5B show the (A) amino acid (SEQ ID NO: 4) and (B) nucleic acid coding sequence (SEQ ID NO: 3) of human Eno1, variant 2 (NCBI Accession No. NM_001201483.1). The human Eno1, variant 2 protein is also referred to as MBP-1.

FIGS. 6A and 6B show fluorescent images of the tissue distribution in mice of (A) a fluorescently-labeled Eno1-G5-PAMAM dendrimer complex and (B) a fluorescently-labeled, muscle targeted Eno-1-G5-PAMAM dendrimer complex.

### DETAILED DESCRIPTION AND PREFERRED EMBODIMENTS

An Eno1 muscle targeted dendrimer complex was prepared. Administration of the Eno1 dendrimer complex in combination with rosiglitazone was shown to attenuate rosiglitazone-induced weight gain in a diabetic mouse model (db/db mice). In addition, administration of the Eno1 dendrimer complex in combination with rosiglitazone more quickly reduced fed blood glucose levels in diabetic mice compared to rosiglitazone alone. The Eno1 muscle targeted dendrimer complex was shown to be effectively targeted to skeletal muscle. These results demonstrate that Eno1 is effective in reducing weight gain, and thus indicate that Eno1 is useful in the treatment of obesity and in reducing weight in a subject suffering from an overweight condition.

### I. DEFINITIONS

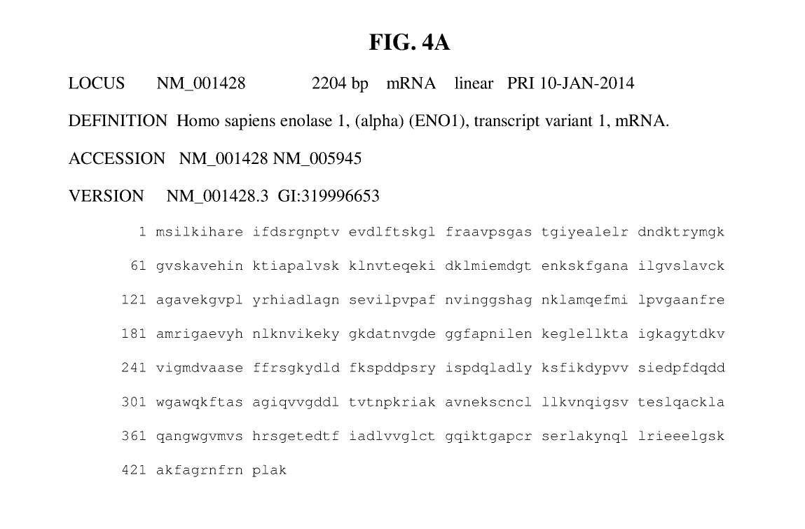

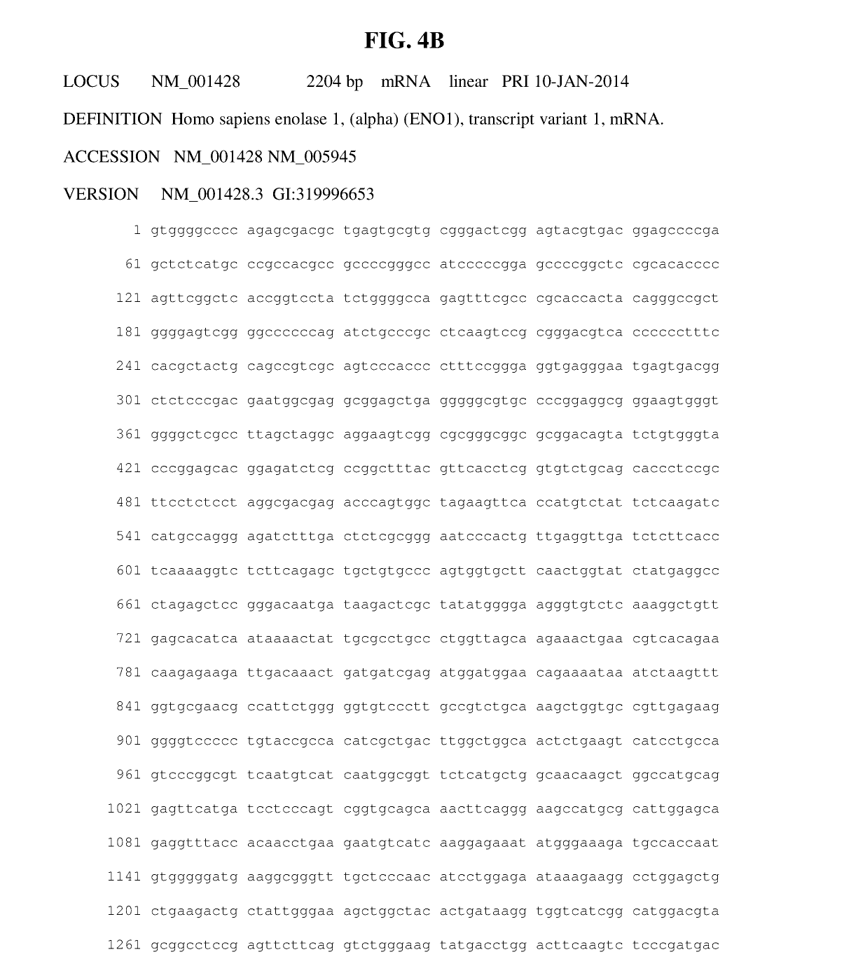

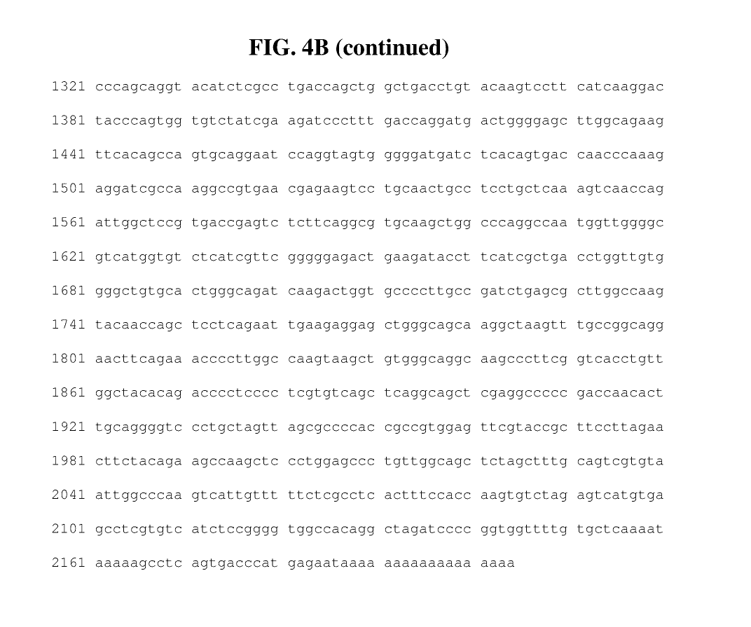

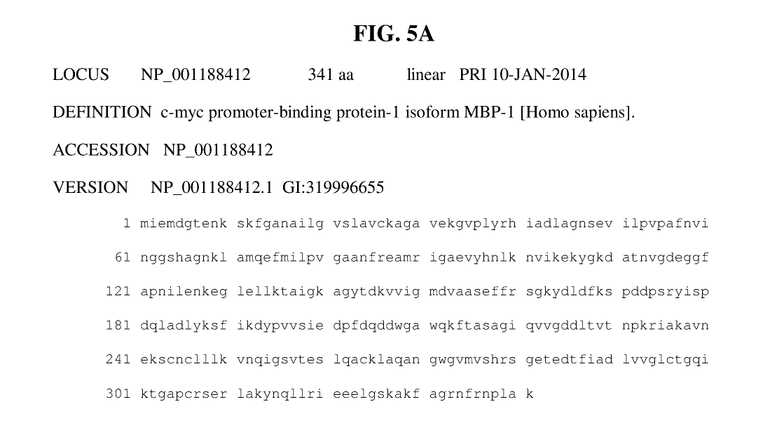

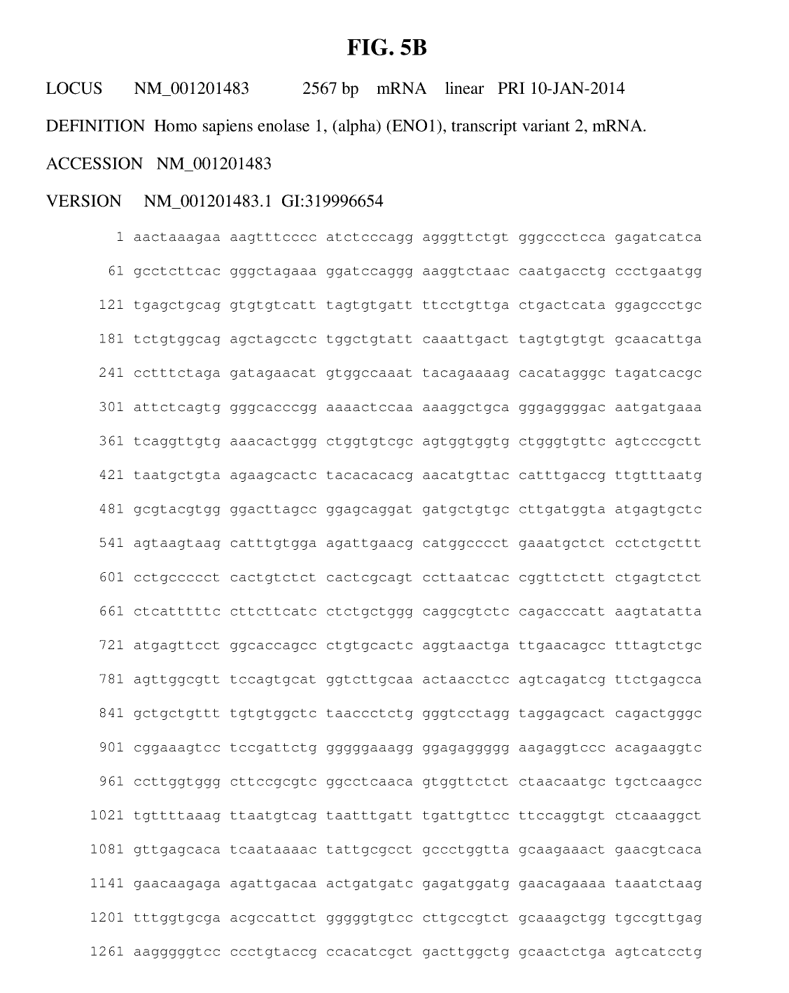

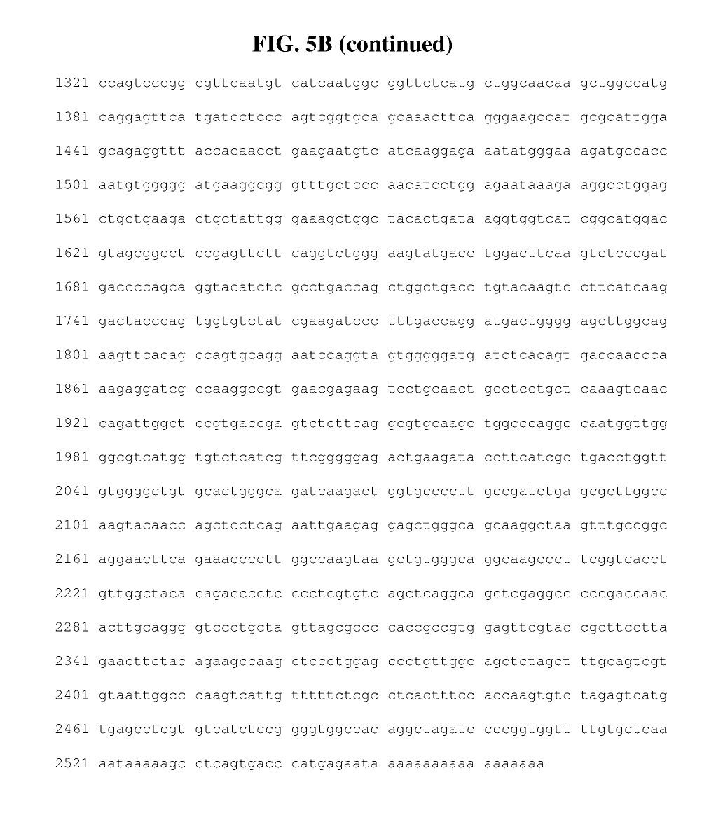

Enolase 1, (alpha), also known as ENO1L, alpha-enolase, enolase-alpha, tau-crystallin, non-neural enolase (NNE), alpha enolase like 1, phosphopyruvate hydratase (PPH), plasminogen-binding protein, MYC promoter-binding protein 1 (MPB1), and 2-phospho-D-glycerate hydro-lyase, is one of three enolase isoenzymes found in mammals. Protein and nucleic acid sequences of human Eno1 isoforms are provided herein in FIGS. 4 and 5. The instant application provides human amino acid and nucleic acid sequences for the treatment of human disease. However, it is understood that the compositions and methods of the invention can be readily adapted for treatment of non-human animals by selection of an Eno1 of the species to be treated. Amino acid and nucleic acid sequences of Eno1 for non-human species are known in the art and can be found, for example, at ncbi.nlm.nih.gov/genbank/. In some embodiments, the Eno1 used in the compositions and methods of the invention is a mammalian Eno1. In a preferred embodiment, the Eno1 is human Eno1.

As used herein, “administration of Eno1” unless otherwise indicated is understood as administration of either Eno1 protein or a nucleic acid construct for expression of Eno1 protein. In certain embodiments the Eno1 protein can include an Eno1 protein fragment or a nucleic acid for encoding an Eno1 protein fragment. In certain embodiments, administration of Eno1 is administration of Eno1 protein. In certain embodiments, administration of Eno1 is administration of Eno1 polynucleotide. Protein and nucleic acid sequences of human Eno1 are provided herein. In certain embodiments, administration of Eno1 comprises administration of the first variant or the second variant of human Eno1. In certain embodiments, administration of Eno1 comprises administration of the first variant and the second variant of human Eno1. In certain embodiments, administration of Eno1 comprises administration of the first variant of human Eno1. In certain embodiments, administration of Eno1 comprises administration of the second variant of human Eno1. In certain embodiments, administration of Eno1 comprises administration of only the first variant of human Eno1. In certain embodiments, administration of Eno1 comprises administration of only the second variant of human Eno1.

In certain embodiments, the fragment of Eno1 comprises at least 10, 20, 30, 40, 50, 60, 70, 80, 90, 100, 150, 200, 250, 300, 350, or 400 amino acid residues. In certain embodiments, the fragment of Eno1 is a nucleic acid comprising at least 50, 100, 150, 200, 250, 300, 350, 400, 450, 500, 600, 700, 800, 900, 1000, 1100 or 1200 nucleotides. In certain embodiments, the fragment of Eno1 is biologically active.

As used herein, “biologically active” refers to an Eno1 molecule or fragment thereof that has at least one activity of an endogenous Eno1 protein. For example, in some embodiments, the biologically active Eno1 molecule or fragment thereof catalyzes the dehydration of 2-phospho-D-glycerate (PGA) to phosphoenolpyruvate (PEP). In some embodiments, the biologically active Eno1 molecule or fragment thereof catalyzes the hydration of PEP to PGA. In some embodiments, the biologically active Eno1 molecule or fragment thereof increases glucose uptake by a cell, for example a muscle cell, preferably a skeletal muscle cell. In some embodiments, the biologically active Eno1 molecule or fragment thereof reduces blood glucose levels, e.g. fed blood glucose levels or blood glucose levels in a glucose tolerance test. In some embodiments, the biologically active Eno1 molecule or fragment thereof binds to Nampt, for example, extracellular Nampt (eNampt).

As used herein, “administration to a muscle”, “delivery to a muscle”, or “delivery to a muscle cell” including a skeletal muscle cell, smooth muscle cell, and the like are understood as a formulation, method, or combination thereof to provide an effective dose of Eno1 to a muscle e.g., a muscle cell, to provide a desired systemic effect, e.g., normalization of blood glucose in a subject with abnormal blood glucose, e.g., by increasing glucose tolerance and/or insulin sensitivity, treating diabetes, treating obesity, reducing body weight, or reducing or preventing body weight gain. In certain embodiments, the Eno1 is formulated for administration directly to, and preferably retention in, muscle. In certain embodiments, the formulation used for administration directly to the muscle (i.e., intramuscular administration) preferably a sustained release formulation of the Eno1 to permit a relatively low frequency of administration (e.g., once per week or less, every other week or less, once a month or less, once every other month or less, once every three months or less, once every four months or less, once every five months or less, once every six months or less). In certain embodiments, the Eno1 is linked to a targeting moiety to increase delivery of the Eno1 to muscle so that the Eno1 need not be delivered directly to muscle (e.g., is delivered subcutaneously or intravenously). It is understood that administration to muscle does not require that the entire dose of Eno1 be delivered to the muscle or into muscle cells. In certain embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or more of the Eno1 is delivered to muscle, preferably skeletal muscle and/or smooth muscle. In certain embodiments, the amount of non-intramuscularly administered muscle-targeted Eno1 delivered to a muscle cell is about 1.2 or more times greater, about 1.3 or more times greater, about 1.4 or more times greater, about 1.5 or more times greater, about 1.75 or more times greater, about 2 or more times greater, 3 or more times greater, 4 or more times greater, 5 or more times greater, or 6 or more times greater than the amount of non-targeted Eno1 delivered to muscle. In certain embodiments, the amount of non-intramuscularly administered muscle-targeted Eno1 delivered to a muscle cell is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% greater than the amount of non-targeted Eno1 delivered to muscle.

In certain embodiments, the Eno1 is delivered to skeletal muscle. In certain embodiments, the Eno1 is delivered to smooth muscle. In certain embodiments, the Eno1 is delivered to skeletal muscle and smooth muscle. In certain embodiments, is delivered preferentially or in greater amount to skeletal muscle as compared to smooth muscle. In certain embodiments, at least 5%, 10%, 15%, 20%, 25%, 30%, 35%, 40%, 45%, 50%, 55%, 60%, 65%, 70%, 75%, 80%, 85%, 90%, 95% or greater of the Eno1 delivered to muscle is delivered to skeletal muscle. In certain embodiments, the Eno1 is not delivered to smooth muscle. Assays to determine the relative targeting of a payload by a targeting moiety are known in the art and provided, for example, in Samoylova et al., 1999, Muscle Nerve, 22:460-466, which is expressly incorporated herein by reference in its entirety.

As used herein, a “muscle targeting moiety” includes a muscle targeting peptide (MTP), for example a skeletal and/or smooth muscle targeting peptide (SMTP). In certain embodiments, the targeting moiety include ligands to bind integrins αvβ5 or αvβ3 integrins. In certain embodiments, the targeting moiety includes a CD-46 ligand. In certain embodiments, the targeting moiety includes an adenovirus peton protein optionally in combination with an adenovirus 35 fiber protein. In certain embodiments, at least 5%, at least 10%, at least 15%, at least 20%, at least 25%, at least 30%, at least 35% of muscle-targeted Eno1 is delivered to muscle, in some embodiments preferably skeletal and/or smooth muscle, by a muscle-targeting moiety. In certain embodiments, the amount of non-intramuscularly administered muscle-targeted Eno1 delivered to a muscle cell is about 1.5 or more times greater, 2 or more times greater, 3 or more times greater, 4 or more times greater, 5 or more times greater, or 6 or more times greater than the amount of non-targeted Eno1 delivered to muscle.

As used herein, a “muscle targeting peptide” or “MTP” is understood as a peptide sequence that increases the delivery of its payload (e.g., Eno1) to a muscle cell, preferably a skeletal and/or smooth muscle cell. MTPs are known in the art and are provided, for example, in U.S. Pat. No. 6,329,501; US Patent Publication No. 20110130346; and Samoylova et al., 1999, Muscle and Nerve 22: 460-466, each of which is expressly incorporated herein by reference in its entirety. In certain embodiments the MTP is a skeletal muscle targeting peptide. A “skeletal muscle targeting peptide” is a peptide sequence that increases the delivery of its payload (e.g., Eno1) to a skeletal muscle cell. In certain embodiments the MTP is a smooth muscle targeting peptide. A “smooth muscle targeting peptide” is a peptide sequence that increases the delivery of its payload (e.g., Eno1) to a smooth muscle cell. In certain embodiments the MTP increases the delivery of its payload (e.g., Eno1) to a skeletal cell and to a smooth muscle cell. In certain embodiments the MTP, e.g., skeletal muscle targeting peptide and/or smooth muscle targeting peptide, does not increase the delivery of its payload to cardiac muscle cell. MTP, e.g., skeletal muscle, targeting peptides include, but are not limited to peptides comprising the following sequences: ASSLNIA (SEQ ID NO: 7); WDANGKT (SEQ ID NO: 8); GETRAPL (SEQ ID NO: 9); CGHHPVYAC (SEQ ID NO: 5); and HAIYPRH (SEQ ID NO: 6). In a preferred embodiment, the MTP comprises the amino acid sequence ASSLNIA (SEQ ID NO: 7).

As used herein, “payload” is understood as a moiety for delivery to a target cell by a targeting moiety. In certain embodiments, the payload is a peptide, e.g., an Eno1 peptide. In certain embodiments, the payload is a nucleic acid, e.g., a nucleic acid encoding an Eno1 peptide. In certain embodiments, the payload further comprises additional components (e.g., dendrimers, liposomes, microparticles) or agents (e.g., therapeutic agents) for delivery with the Eno1 payload to the target cell.

As used herein, a “linker” is understood as a moiety that juxtaposes a targeting moiety and a payload in sufficiently close proximity such that the payload is delivered to the desired site by the targeting moiety. In certain embodiments, the linker is a covalent linker, e.g., a cross-linking agent including a reversible cross-linking agent; a peptide bond, e.g., wherein the payload is a protein co-translated with the targeting moiety. In certain embodiments, the linker is covalently joined to one of the payload or the targeting moiety and non-covalently linked to the other. In certain embodiments, the linker comprises a dendrimer. In certain embodiments, the dendrimer is covalently linked to the targeting moiety and non-covalently linked to the payload, e.g., Eno1. In certain embodiments, the linker is a liposome or a microparticle, and the targeting moiety is exposed on the surface of the liposome and the payload, e.g., Eno1 is encapsulated in the liposome or microparticle. In certain embodiments, the linker and the Eno1 are present on the surface of the microparticle linker. In certain embodiments, the targeting moiety is present on the surface of a virus particle and the payload comprises a nucleic acid encoding Eno1.

As used herein, “linked”, “operably linked”, “joined” and the like refer to a juxtaposition wherein the components described are present in a complex permitting them to function in their intended manner. The components can be linked covalently (e.g., peptide bond, disulfide bond, non-natural chemical linkage), through hydrogen bonding (e.g., knob-into-holes pairing of proteins, see, e.g., U.S. Pat. No. 5,582,996; Watson-Crick nucleotide pairing), or ionic binding (e.g., chelator and metal) either directly or through linkers (e.g., peptide sequences, typically short peptide sequences; nucleic acid sequences; or chemical linkers, including the use of linkers for attachment to higher order or larger structures including microparticles, beads, or dendrimers). As used herein, components of a complex can be linked to each other by packaging in and/or on a liposome and/or dendrimer wherein some of the components of the complex can be attached covalently and some non-covalently. Linkers can be used to provide separation between active molecules so that the activity of the molecules is not substantially inhibited (less than 10%, less than 20%, less than 30%, less than 40%, less than 50%) by linking the first molecule to the second molecule. Linkers can be used, for example, in joining Eno1 to a targeting moiety. As used herein, molecules that are linked, but not covalently joined, have a binding affinity (Kd) of less than 10⁻³, 10⁻⁴, 10⁻⁵, 10⁻⁶, 10, 10⁻⁸, 10, 10⁻¹⁰, 10⁻¹¹, or 10⁻¹², or any range bracketed by those values (e.g., between 10⁻³ and 10⁻⁵, or between 10⁻⁵ and 10⁻⁸) for each other under conditions in which the reagents of the invention are used, i.e., typically physiological conditions.

In certain embodiments, the payload (e.g. Eno1) and the targeting moiety are present in a complex at about a 1:1 molar ratio. In certain embodiments, the targeting moiety is present in a complex with a molar excess of the payload (e.g. Eno1). In certain embodiments, the ratio of payload (e.g. Eno1) to targeting moiety is about 0.1:1, about 0.2:1, about 0.3:1, about 0.4:1, about 0.5:1, about 0.6:1, about 0.7:1, about 0.8:1, about 0.9:1, about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, or about 20:1.

A “dendrimer” is a polymeric molecule composed of multiple branched monomers that emanate radially from a central core. Due to the structure and synthetic methods used to generate dendrimers, the products from dendrimer synthesis are theoretically monodisperse. When the core of a dendrimer is removed, a number of identical fragments called dendrons remain with the number of dendrons dependent on the multiplicity of the central core. The number of branch points encountered upon moving outward from the core to the periphery defines its generation, e.g., G-1, G-2, G-3, etc., with dendrimers of higher generations being larger, more branched, and having more end groups than dendrimers of lower generations. As used herein, a dendrimer is preferably a pharmaceutically acceptable dendrimer. Compositions comprising Eno1 and a dendrimer are described, for example, in US 2015/0361409, which is incorporated by reference herein in its entirety.

As used herein, the term “subject” refers to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human and may be referred to as a patient.

As used herein, the terms “treat,” “treating” or “treatment” refer, preferably, to an action to obtain a beneficial or desired clinical result including, but not limited to, alleviation or amelioration of one or more signs or symptoms of a disease or condition, diminishing the extent of disease, stability (i.e., not worsening) state of disease, amelioration or palliation of the disease state. A “therapeutically effective amount” is that amount sufficient to treat a disease in a subject. A therapeutically effective amount can be administered in one or more administrations.

As used herein, the term “therapeutic treatment that induces weight gain” refers to any method of drug for the treatment of a disorder that results in increased body mass in a subject. Increased body mass can be relative to a subject or population of subjects that does not receive the treatment, or relative to the body mass of subject or population of subjects prior to treatment. Therapeutic treatments that induce weight gain include, but are not limited to, therapeutic agents for the treatment of diabetes, antipsychotic agents, antidepressants, mood stabilizers, anticonvulsants, steroid hormones, prednisone beta-blockers, oral contraceptives, antihistamines, HIV antiretroviral drugs, antiseizure and antimigraine drugs, protease inhibitors, antihyperlipemic agents, hypotensive or antihypertensive agents, anti-obesity agents, diuretics, chemotherapeutic agents, immunotherapeutic agents, and immunosuppressive agents.

A number of treatments for type 2 diabetes are known in the art including both drug and behavioral interventions. Drugs for treatment of type 2 diabetes include, but are not limited to GLP-1, meglitinides (repaglinide (Prandin) and nateglinide (Starlix); sulfonylureas (glipizide (Glucotrol), glimepiride (Amaryl), and glyburide (DiaBeta, Glynase)); Dipeptidy peptidase-4 (DPP-4) inhibitors (saxagliptin (Onglyza), sitagliptin (Januvia), and linagliptin (Tradjenta)); biguanides (metformin (Fortamet, Glucophage)); thiazolidinediones (rosiglitazone (Avandia) and pioglitazone (Actos)); and alpha-glucosidase inhibitors (acarbose (Precose) and miglitol (Glyset)). Insulins are typically used only in treatment of later stage type 2 diabetes and include rapid-acting insulin (insulin aspart (NovoLog), insulin glulisine (Apidra), and insulin lispro (Humalog)); short-acting insulin (insulin regular (Humulin R, Novolin R)); intermediate-acting insulin (insulin NPH human (Humulin N, Novolin N)), and long-acting insulin (insulin glargine (Lantus) and insulin detemir (Levemir)). Treatments for diabetes can also include behavior modification including exercise and weight loss which can be facilitated by the use of drugs or surgery. Treatments for elevated blood glucose and diabetes can be combined. For example, drug therapy can be combined with behavior modification therapy.

The terms “administer”, “administering” or “administration” include any method of delivery of a pharmaceutical composition or agent into a subject's system or to a particular region in or on a subject. In certain embodiments, the agent is administered enterally or parenterally. In certain embodiments of the invention, an agent is administered intravenously, intramuscularly, subcutaneously, intradermally, intranasally, orally, transcutaneously, or mucosally. In certain preferred embodiments, an agent is administered by injection or infusion, e.g., intravenously, intramuscularly, subcutaneously. In certain embodiments, administration includes the use of a pump. In certain embodiments, the agent is administered locally or systemically. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, etc.

As used herein, the term “co-administering” refers to administration of Eno1 prior to, concurrently or substantially concurrently with, subsequently to, or intermittently with the administration of an additional agent, e.g., for the treatment of diabetes, pre-diabetes, glucose intolerance, insulin resistance, obesity, overweight or weight gain. The Eno1 formulations provided herein, can be used in combination therapy with at least one other therapeutic agent for the treatment of diabetes, pre-diabetes, glucose intolerance, insulin resistance, obesity, overweight or weight gain. Eno1 and/or pharmaceutical formulations thereof and the other therapeutic agent can act additively or, more preferably, synergistically. In one embodiment, Eno1 and/or a formulation thereof is administered concurrently with the administration of another therapeutic agent for the treatment of diabetes, pre-diabetes, glucose intolerance, insulin resistance, obesity, overweight or weight gain. In another embodiment, Eno1 and/or a pharmaceutical formulation thereof is administered prior or subsequent to administration of another therapeutic agent for the treatment of diabetes, pre-diabetes, glucose intolerance, insulin resistance, obesity, overweight or weight gain.

“Obesity” or “obese” refers to the condition where a patient has a body mass index (BMI) equal to or greater than 30 kg/m². “Visceral obesity” refers to a waist to hip ration of 1.0 in male patients and 0.8 in female patients. In another aspect, visceral obesity defines the risk for insulin resistance and the development of pre-diabetes.

“Overweight” or “subject afflicted with an overweight condition” refers to a patient with a body mass index (BMI) greater than or equal to 25 kg/m² and less than 30 kg/m². “Weight gain” refers to the increase in body weight in relationship to behavioral habits or addictions, e.g., overeating or gluttony, smoking cessation, or in relationship to biological (life) changes, e.g., weight gain associated with aging in men and menopause in women or weight gain after pregnancy, or as a side effect of a therapeutic treatment, e.g., a treatment known to induce or cause weight gain.

The articles “a”, “an” and “the” are used herein to refer to one or to more than one (i.e. to at least one) of the grammatical object of the article unless otherwise clearly indicated by contrast. By way of example, “an element” means one element or more than one element.

The term “including” is used herein to mean, and is used interchangeably with, the phrase “including but not limited to”.

The term “or” is used herein to mean, and is used interchangeably with, the term “and/or,” unless context clearly indicates otherwise.

The term “such as” is used herein to mean, and is used interchangeably, with the phrase “such as but not limited to”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

The recitation of a listing of chemical group(s) in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Reference will now be made in detail to preferred embodiments of the invention. While the invention will be described in conjunction with the preferred embodiments, it will be understood that it is not intended to limit the invention to those preferred embodiments. To the contrary, it is intended to cover alternatives, modifications, and equivalents as may be included within the spirit and scope of the invention as defined by the appended claims.

### II. OBESITY AND DIABETES

Obesity (commonly defined as a Body Mass Index of approximately >30 kg/m2) is often associated with a variety of pathologic conditions such as hyperinsulinemia, insulin resistance, diabetes, hypertension, and dyslipidemia. Each of these conditions contributes to the risk of cardiovascular disease.

Along with insulin resistance, hypertension, and dyslipidemia, obesity is considered to be a component of the Metabolic Syndrome (also known as Syndrome X) which together synergize to potentiate cardiovascular disease. More recently, the U.S. National Cholesterol Education Program has classified Metabolic Syndrome as meeting three out of the following five criteria: fasting glucose level of at least 110 mg/dl, plasma triglyceride level of at least 150 mg/dl (hypertriglycerdemia), HDL cholesterol below 40 mg/dl in men or below 50 mg/dl in women, blood pressure at least 130/85 mm Hg (hypertension), and central obesity, with central obesity being defined as abdominal waist circumference greater than 40 inches for men and greater than 35 inches for women.

Diabetes mellitus (DM), often simply referred to as diabetes, is a group of metabolic diseases in which a person has high blood sugar, either because the body does not produce enough insulin or because cells do not respond to the insulin that is produced. This high blood sugar produces the classical symptoms of polyuria (frequent urination), polydipsia (increased thirst), and polyphagia (increased hunger).

Type 2 diabetes results from insulin resistance, a condition in which cells fail to use insulin properly, sometimes combined with an absolute insulin deficiency. The defective responsiveness of body tissues to insulin is believed, at least in part, to involve the insulin receptor. However, the specific defects are not known.

In the early stage of type 2 diabetes, the predominant abnormality is reduced insulin sensitivity. At this stage, hyperglycemia can be reversed by a variety of measures and medications that improve insulin sensitivity or reduce glucose production by the liver. Prediabetes indicates a condition that occurs when a person's blood glucose levels are higher than normal but not high enough for a diagnosis of type 2 diabetes.

Type 2 diabetes is due to insufficient insulin production from beta cells in the setting of insulin resistance. Insulin resistance, which is the inability of cells to respond adequately to normal levels of insulin, occurs primarily within the muscles, liver, and fat tissue. In the liver, insulin normally suppresses glucose release. However in the setting of insulin resistance, the liver inappropriately releases glucose into the blood. The proportion of insulin resistance verses beta cell dysfunction differs among individuals with some having primarily insulin resistance and only a minor defect in insulin secretion and others with slight insulin resistance and primarily a lack of insulin secretion.

Other potentially important mechanisms associated with type 2 diabetes and insulin resistance include: increased breakdown of lipids within fat cells, resistance to and lack of incretin, high glucagon levels in the blood, increased retention of salt and water by the kidneys, and inappropriate regulation of metabolism by the central nervous system. However not all people with insulin resistance develop diabetes, since an impairment of insulin secretion by pancreatic beta cells is also required.

Type 1 diabetes results from the body's failure to produce insulin, and presently requires treatment with injectable insulin. Type 1 diabetes is characterized by loss of the insulin-producing beta cells of the islets of Langerhans in the pancreas, leading to insulin deficiency. Most affected people are otherwise healthy and of a healthy weight when onset occurs. Sensitivity and responsiveness to insulin are usually normal, especially in the early stages. However, particularly in late stages, insulin resistance can occur, including insulin resistance due to immune system clearance of administered insulin.

### III. ENOLASE 1

Enolase 1, (alpha), also known as ENO1L, alpha-enolase, enolase-alpha, tau-crystallin, non-neural enolase (NNE), alpha enolase like 1, phosphopyruvate hydratase (PPH), plasminogen-binding protein, MYC promoter-binding protein 1 (MPB1), and 2-phospho-D-glycerate hydro-lyase, is one of three enolase isoenzymes found in mammals. Each isoenzyme is a homodimer composed of 2 alpha, 2 gamma, or 2 beta subunits, and functions as a glycolytic enzyme. Alpha-enolase in addition, functions as a structural lens protein (tau-crystallin) in the monomeric form. Alternative splicing of this gene results in a shorter isoform that has been shown to bind to the c-myc promoter and function as a tumor suppressor. Several pseudogenes have been identified, including one on the long arm of chromosome 1. Alpha-enolase has also been identified as an autoantigen in Hashimoto encephalopathy. Further information regarding human Eno1 can be found, for example, in the NCBI gene database under Gene ID No. 2023 (see, www.ncbi.nlm.nih.gov/gene/2023, incorporated herein by reference in the version available on the date of filing this application).

### 1. Eno1 Variants

Two isoforms of human Eno1 are known. Protein and mRNA sequences of Homo sapiens enolase 1, (alpha) (ENO1), transcript variant 1, mRNA can be found at GenBank Accession No. NM_001428 (see ncbi.nlm.nih.gov/nuccore/NM_001428.3, which is incorporated by reference in the version available on the date of filing the instant application). This variant encodes the longer isoform, which is localized to the cytosol, and has alpha-enolase activity. It has been reported that the monomeric form of this isoform functions as a structural lens protein (tau-crystallin), and the dimeric form as an enolase. In a preferred embodiment of the invention, Eno1 is the transcript variant 1 of Eno1.

Protein and mRNA sequences of the Homo sapiens enolase 1, (alpha) (ENO1), transcript variant 2, mRNA can be found at GenBank Accession No. NM_001201483 (see www.ncbi.nlm.nih.gov/nuccore/NM_001201483.1, which is incorporated by reference in the version available on the date of filing the instant application). This variant differs at the 5′ end compared to variant 1, and initiates translation from an in-frame downstream start codon, resulting in a shorter isoform (MBP-1). This isoform is localized to the nucleus, and functions as a transcriptional repressor of c-myc protooncogene by binding to its promoter. In certain embodiments of the invention, Eno1 is the transcript variant 2 of Eno1.

Several additional variants of the Eno1 protein have been described, for example, in the UniProtKB/Swiss-Prot database under Accession No. P06733. Examples of Eno1 protein variants are shown in Table 1 below.

TABLE 1
Eno1 variants.

| | AA residue | Modification |
|---|---|---|
| AA modification | 2 | N-acetylserine |
| AA modification | 5 | N6-acetyllysine |
| AA modification | 44 | Phosphotyrosine |
| AA modification | 60 | N6-acetyllysine; alternate |
| AA modification | 60 | N6-succinyllysine; alternate |
| AA modification | 64 | N6-acetyllysine |
| AA modification | 71 | N6-acetyllysine |
| AA modification | 89 | N6-acetyllysine; alternate |
| AA modification | 89 | N6-succinyllysine; alternate |
| AA modification | 92 | N6-acetyllysine |
| AA modification | 126 | N6-acetyllysine |
| AA modification | 193 | N6-acetyllysine |
| AA modification | 199 | N6-acetyllysine |
| AA modification | 202 | N6-acetyllysine |
| AA modification | 228 | N6-acetyllysine; alternate |
| AA modification | 228 | N6-succinyllysine; alternate |
| AA modification | 233 | N6-acetyllysine; alternate |
| AA modification | 233 | N6-malonyllysine; alternate |
| AA modification | 254 | Phosphoserine |
| AA modification | 256 | N6-acetyllysine |
| AA modification | 263 | Phosphoserine |
| AA modification | 272 | Phosphoserine |
| AA modification | 281 | N6-acetyllysine |
| AA modification | 285 | N6-acetyllysine |
| AA modification | 287 | Phosphotyrosine |
| AA modification | 335 | N6-acetyllysine |
| AA modification | 343 | N6-acetyllysine |
| AA modification | 406 | N6-acetyllysine |
| AA modification | 420 | N6-acetyllysine; alternate |
| AA modification | 420 | N6-malonyllysine; alternate |
| AA modification | 420 | N6-succinyllysine; alternate |
| Natural variant | 177 | N → K. |
| | | Corresponds to variant rs11544513 |
| | | [ dbSNP \| Ensembl ]. |
| Natural variant | 325 | P → Q. |
| | | Corresponds to variant rs11544514 |
| | | [ dbSNP \| Ensembl ]. |
| Mutagenesis | 94 | M → I: MBP1 protein production. No MBP1 |
| | | protein production; when associated with |
| | | I-97. |
| Mutagenesis | 97 | M → I: MBP1 protein production. No MBP1 |
| | | protein production; when associated with |
| | | I-94. |
| Mutagenesis | 159 | Dramatically decreases activity levels |
| Mutagenesis | 168 | Dramatically decreases activity levels |
| Mutagenesis | 211 | Dramatically decreases activity levels |
| Mutagenesis | 345 | Dramatically decreases activity levels |
| Mutagenesis | 384 | L → A: Loss of transcriptional repression |
| | | and cell growth inhibition; |
| | | when associated with A-388. |
| Mutagenesis | 388 | L → A: Loss of transcriptional repression |
| | | and cell growth inhibition; |
| | | when associated with A-384. |
| Mutagenesis | 396 | Dramatically decreases activity levels |

In certain embodiments of the invention, Eno1 is one of the variants listed in Table 1.

In some embodiments, the Eno1 comprises a nucleic acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the nucleic acid sequence of SEQ ID NO: 1 or SEQ ID NO: 3.

In some embodiments, the Eno1 consists of a nucleic acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the nucleic acid sequence of SEQ ID NO: 1 or SEQ ID NO: 3.

In some embodiments, the Eno1 comprises an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the amino acid sequence of SEQ ID NO: 2 or SEQ ID NO: 4.

In some embodiments, the Eno1 consists of an amino acid sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the amino acid sequence of SEQ ID NO: 2 or SEQ ID NO: 4.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percentage sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters. For local alignments, the Smith-Waterman algorithm is particularly useful (Smith T F, Waterman M S (1981) J. Mol. Biol. 147(1); 195-7).

The term “hybridization” as defined herein is a process wherein substantially homologous complementary nucleotide sequences anneal to each other. The term “stringency” refers to the conditions under which a hybridization takes place. The stringency of hybridization is influenced by conditions such as temperature, salt concentration, ionic strength and hybridization buffer composition. Generally, low stringency conditions are selected to be about 30° C. lower than the thermal melting point (Tₘ) for the specific sequence at a defined ionic strength and pH. Medium stringency conditions are when the temperature is 20° C. below Tₘ, and high stringency conditions are when the temperature is 10° C. below Tₘ. High stringency hybridization conditions are typically used for isolating hybridizing sequences that have high sequence similarity to the target nucleic acid sequence. However, nucleic acids may deviate in sequence and still encode a substantially identical polypeptide, due to the degeneracy of the genetic code. Therefore medium stringency hybridization conditions may sometimes be needed to identify such nucleic acid molecules.

For example, typical high stringency hybridization conditions for DNA hybrids longer than 50 nucleotides encompass hybridization at 65° C. in 1×SSC or at 42° C. in 1×SSC and 50% formamide, followed by washing at 65° C. in 0.3×SSC. Examples of medium stringency hybridization conditions for DNA hybrids longer than 50 nucleotides encompass hybridization at 50° C. in 4×SSC or at 40° C. in 6×SSC and 50% formamide, followed by washing at 50° C. in 2×SSC. 1×SSC is 0.15M NaCl and 15 mM sodium citrate; the hybridization solution and wash solutions may additionally include 5×Denhardt's reagent, 0.5-1.0% SDS, 100 μg/ml denatured, fragmented salmon sperm DNA, 0.5% sodium pyrophosphate. In a preferred embodiment high stringency conditions mean hybridization at 65° C. in 0.1×SSC comprising 0.1% SDS and optionally 5×Denhardt's reagent, 100 μg/ml denatured, fragmented salmon sperm DNA, 0.5% sodium pyrophosphate, followed by the washing at 65° C. in 0.3×SSC. For the purposes of defining the level of stringency, reference can be made to Sambrook et al. (2001) Molecular Cloning: a laboratory manual, 3rd Edition, Cold Spring Harbor Laboratory Press, CSH, New York or to Current Protocols in Molecular Biology, John Wiley & Sons, N.Y. (1989 and yearly updates).

In some embodiments, the Eno1 hybridizes to the complement of the nucleic acid sequence of SEQ ID NO: 1 or SEQ ID NO: 3 under high stringency hybridization conditions or medium stringency hybridization conditions as defined above.

### 2. Eno1 Activity

Eno1 is a key glycolytic enzyme that catalyzes the dehydratation of 2-phospho-D-glycerate (PGA) to phosphoenolpyruvate (PEP) in the last steps of the catabolic glycolytic pathway (see Diaz-Ramos et al., 2012, J Biomed Biotechnol. 2012: 156795). Enolase enzymes catalyse the dehydration of PGA to PEP in the Emden Mayerhoff-Parnas glycolytic pathway (catabolic direction). In the anabolic pathway (reverse reaction) during gluconeogenesis, Eno1 catalyzes hydration of PEP to PGA. Accordingly Eno1 is also known as phosphopyruvate hydratase. Metal ions are cofactors impairing the increase of enolase activity; hence Eno1 is also called metal-activated metalloenzyme. Magnesium is a natural cofactor causing the highest activity and is required for the enzyme to be catalytically active. The relative activation strength profile of additional metal ions involved in the enzyme activity appears in the following order Mg²⁺>Zn²⁺>Mn²⁺>Fe(II)²⁺>Cd²⁺>Co^{2±}, Ni^{2±}, Sm^{3±}, Tb³⁺ and most other divalent metal ions. In reactions catalyzed by enolases, the alpha-proton from a carbon adjacent to a carboxylate group of PGA, is abstracted, and PGA is conversed to enolate anion intermediate. This intermediate is further processed in a variety of chemical reactions, including racemization, cycloisomerization and beta-elimination of either water or ammonia (see Atlas of Genetics and Cytogenetics in Oncology and Haematology database, atlasgeneticsoncology.org/Genes/GC_ENO1.html).

Enzymatically active enolase exists in a dimeric (homo- or heterodimers) form and is composed of two subunits facing each other in an antiparallel fashion. The crystal structure of enolase from yeast and human has been determined and catalytic mechanisms have been proposed (Diaz-Ramos et al., cited above). The five residues that participate in catalytic activity of this enzyme are highly conserved throughout evolution. Studies in vitro revealed that mutant enolase enzymes that differ at positions Glu168, Glu211, Lys345, Lys396 or His159, demonstrate dramatically decreased activity levels. An integral and conserved part of enolases are two Mg2+ ions that participate in conformational changes of the active site of enolase and enable binding of a substrate or its analogues (Atlas of Genetics and Cytogenetics in Oncology database, cited above). In certain embodiments, the compositions of the invention comprise a metal ion cofactor. The metal ion cofactor can provide increased stability of the Eno1 in the composition and/or increased activity of the Eno1 in vivo. In one embodiment, the metal ion cofactor is divalent. In one embodiment, the divalent metal ion cofactor is Mg²⁺, Zn²⁺, Mn²⁺, Fe(II)^{2±}, Cd²⁺, Co²⁺, or Ni^{2±}. In one embodiment, the metal ion cofactor is trivalent, e.g. Sm³⁺ or Tb3⁺.

Eno1 activity may be determined, for example, using the pyruvate kinase (PK)/lactate dehydrogenase (LDH) assay. The reaction for this enolase assay is shown below.

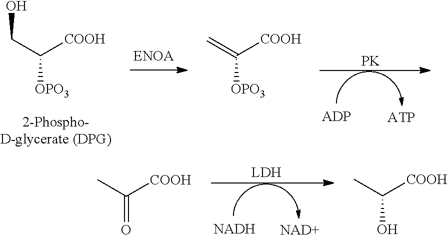

The rate of reaction of NADH to NAD conversion may be determined by measuring the decrease of fluorescence of NADH, for example by using a PTI Quantamaster 40 spectrophotometer from Photon Technology International, Inc. (pti-nj.com). Kits for measuring Eno1 activity by a colorimetric pyruvate kinase/lactate dehydrogenase assay are also commercially available, for example, from ABCAM (Cambridge, Mass.; Cat. No. ab117994). The ABCAM Eno1 activity assay is further described in Example 5 of US 2015/0361409, which is incorporated by reference herein in its entirety.

Eno1 activity may also be determined by measuring the effect of Eno1 on glucose uptake in human skeletal muscle myotubes (HSMM). “Increasing glucose flux” as used herein is understood as increasing at least one or more of (1) delivery of glucose to muscle, (2) transport of glucose into the muscle, and (3) phosphorylation of glucose within the muscle. In particular embodiments, increasing glucose flux includes increasing glycolytic activity or mitochondrial free fatty acid oxidation in a muscle cell. The effect of Eno1 on glucose uptake can be measured by using methods known in the art and as described for example in Example 2 of US 2015/0361409, which is incorporated by reference herein in its entirety.

The regulation of muscle glucose uptake involves a three-step process consisting of: (1) delivery of glucose to muscle, (2) transport of glucose into the muscle by the glucose transporter GLUT4 and (3) phosphorylation of glucose within the muscle by a hexokinase (HK). The physiological regulation of muscle glucose uptake requires that glucose travels from the blood to the interstitium to the intracellular space and is then phosphorylated to G6P. Blood glucose concentration, muscle blood flow and recruitment of capillaries to muscle determine glucose movement from the blood to the interstitium. Plasma membrane GLUT4 content controls glucose transport into the cell. Muscle hexokinase (HK) activity, cellular HK compartmentalization and the concentration of the HK inhibitor, G6P, determine the capacity to phosphorylate glucose. These three steps—delivery, transport and phosphorylation of glucose—comprise glucose flux, and all three steps are important for glucose flux control. However steps downstream of glucose phosphorylation may also affect glucose uptake. For example, acceleration of glycolysis or glycogen synthesis could reduce G6P, increase HK activity, increase the capacity for glucose phosphorylation and potentially stimulate muscle glucose uptake. Wasserman et al., 2010, J Experimental Biology, Vol. 214, pp. 254-262.

In certain embodiments, the Eno1 or the fragment thereof has at least 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 300%, 400% or 500% of the activity of a purified endogenous human Eno1 polypeptide. In certain embodiments, the activity of the Eno1, the fragment thereof, and the purified endogenous human Eno1 polypeptide are determined by the pyruvate kinase/lactate dehydrogenase assay or the HSMM glucose uptake assay described above.

In certain embodiments, the Eno1 polypeptide in complex with a dendrimer as described herein has at least 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 300%, 400% or 500% of the activity of a purified endogenous Eno1 polypeptide that is not in complex with a dendrimer. In certain embodiments, the activity of the Eno1 polypeptide in complex with a dendrimer and the activity of the purified endogenous Eno1 polypeptide that is not in complex with a dendrimer are determined by the pyruvate kinase/lactate dehydrogenase assay or the HSMM glucose uptake assay described above.

In certain embodiments the Eno1 polypeptide in complex with a dendrimer and a targeting moiety, e.g., targeting peptide, as described herein has at least 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 300%, 400% or 500% of the activity of a purified endogenous ENO1 polypeptide that is not in complex with a dendrimer or a targeting peptide. In certain embodiments the activity of the Eno1 polypeptide in complex with a dendrimer and a targeting peptide and the activity of the purified endogenous ENO1 polypeptide that is not in complex with a dendrimer or a targeting peptide are determined by the pyruvate kinase/lactate dehydrogenase assay or the HSMM glucose uptake assay described above.

In one embodiment, the Eno1 or the fragment thereof present in the composition of the invention, wherein the composition comprises a metal ion cofactor (e.g., a divalent metal ion cofactor, e.g., Mg^{2±}, Zn^{2±}, Mn^{2±}, Fe(II)^{2±}, Cd^{2±}, Co^{2±}, or Ni^{2±}, or a trivalent metal ion cofactor, e.g. Sm³⁺ or Tb3⁺) has at least 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 300%, 400% or 500% of the activity of a purified endogenous human Eno1 polypeptide. In certain embodiments, the activity of the Eno1 or the fragment thereof in the composition comprising a metal ion cofactor as described above and the activity of the purified endogenous human Eno1 polypeptide are determined by the pyruvate kinase/lactate dehydrogenase assay or the HSMM glucose uptake assay described above.

### IV. Targeted Drug Delivery

Delivery of drugs or active agents (e.g., Eno1 or a fragment thereof) to their site of action can increase the therapeutic index by reducing the amount of drug required to provide the desired systemic effect. Drugs can be delivered to the site of action by administration of the drug to the target tissue using a method or formulation that will limit systemic exposure, e.g., intramuscular injection, intrasinovial injection, intrathecal injection, intraocular injection. A number of the sustained delivery formulations discussed herein are for intramuscular administration and provide local delivery to muscle tissue. Alternatively, targeting moieties can be associated with or linked to therapeutic payloads for administration to the target site. Targeting moieties can include any of a number of moieties that bind to specific cell types.

### 1. Targeting Moieties

Certain embodiments of the invention include the use of targeting moieties including, without limitation, relatively small peptides (e.g., 25 amino acids or less, 20 amino acids or less, 15 amino acids or less, 10 amino acids or less), muscle targeting peptides (MTP) including smooth muscle and/or skeletal muscle targeting peptides, αvβ3 integrin ligands (e.g., RGD peptides and peptide analogs), αvβ5 integrin ligands, or CD46 ligands as discussed above. It is understood that such peptides can include one or more chemical modifications to permit formation of a complex with Eno1, to modify pharmacokinetic and/or pharmacodynamic properties of the peptides. In certain embodiments, the targeting moiety can be a small molecule, e.g., RGD peptide mimetics. In certain embodiments, the targeting moiety can include a protein and optionally a fiber protein from an adenovirus 35. In certain embodiments, the viral proteins are present on a virus particle. In certain embodiments, the viral proteins are not present on a viral particle. In certain embodiments, the targeting moiety can be an antibody, antibody fragment, antibody mimetic, or T-cell receptor.

### 2. Targeted Complexes

Targeted Eno1 complexes can be administered by a route other than intramuscular injection (e.g., subcutaneous injection, intravenous injection) while providing delivery of the Eno1 to muscle. Targeted complexes can include one or more targeting moieties attached either directly or indirectly to Eno1. Formation of the targeted complex does not substantially or irreversibly inhibit the activity of Eno1 and its effect on normalizing blood glucose levels and insulin response, treating obesity or reducing body weight or reducing weight gain. In certain embodiments, use of a targeted complex can reduce the total amount of Eno1 required to provide an effective dose. Some exemplary, non-limiting, embodiments of targeted complexes are discussed below.

In certain embodiments, the payload and the targeting moiety are present in a complex at about a 1:1 molar ratio. In certain embodiments, the targeting moiety is present in a complex with a molar excess of the payload (e.g., 2:1, 3:1, 4:1, 5:1, 6:1, 7:1; 8:1, 9:1, 10:1, 11:1, 12:1, 13:1, 14:1, 15:1, 16:1, 17:1; 18:1, 19:1, 20:1, 21:1, 22:1, 23:1, 24:1, 25:1, 26:1, 27:1; 28:1, 29:1, 30:1, or more; or any range bracketed by any two values). In certain embodiments, the payload to targeting moiety is about 1:5-1:15; about 1:7-1:13, about 1:8-1:12.

It is understood that the compositions and methods of the invention include the administration of more than one, i.e., a population of, targeting moiety-payload complexes. Therefore, it is understood that the number of targeting moieties per payload can represent an average number of targeting moieties per payload in a population of complexes. In certain embodiments, at least 70% of the complexes have the selected molar ratio of targeting moieties to payload. In certain embodiments, at least 75% of the complexes have the selected molar ratio of targeting moieties to payload. In certain embodiments, at least 80% of the complexes have the selected molar ratio of targeting moieties to payload. In certain embodiments, at least 85% of the complexes have the selected molar ratio of targeting moieties to payload. In certain embodiments, at least 90% of the complexes have the selected molar ratio of targeting moieties to payload.

a. Linkers

A number of chemical linkers are known in the art and available from commercial sources (e.g., Pierce Thermo Fisher Scientific Inc., see, e.g., www.piercenet.com/cat/crosslinking-reagents). Such agents can be used to chemically link, reversibly or irreversibly, one or more targeting moieties to Eno1. Linkers can also be used to attach targeting moieties and Eno1 to a structure, e.g., microparticle, dendrimer, rather than attaching the targeting moiety directly to Eno1. In certain embodiments, the linker attaching Eno1 to the targeted complex is reversible so that the Eno1 is released from the complex after administration, preferably substantially at the muscle.

b. Peptide Bonds

As used herein, targeted complexes can include the translation of Eno1 with a peptide targeting moiety. Methods to generate expression constructs including an amino acid sequence for targeting Eno1 is well within the ability of those of skill in the art.

c. Liposomes

Liposomal delivery systems are known in the art including formulations to limit systemic exposure, thereby reducing systemic exposure and off target effects. For example, Doxil® is a composition in which doxorubicin encapsulated in long-circulating pegylated liposomes that further comprise cholesterol for treatment of certain types of cancer. Various liposomal formulations of amphotericin B including Ambisome®, Abelcet®, and Amphotec® are formulated for intravenous administration in liposomes or a lipid complex containing various phospholipids, cholesterol, and cholesteryl sulfate. Visudine® is verteporfin formulated as a liposome in egg phosphotidyl glycerol and DMPC for intravenous administration. Liposomal formulations are also known for intramuscular injection. Epaxal® is an inactivated hepatitis A virus and Inflexal V® is an inactivated hemaglutinine of influenza virus strains A and B. Both viral preparations are formulated in combinations of DOPC and DOPE. Such liposomes, or other physiologically acceptable liposomes, can be used for the packaging of Eno1 and subsequent surface decoration with targeting moieties to delivery Eno1 to the muscle. Additional moieties to modulate intracellular trafficking of the liposome can also be included. Upon uptake of the liposome into the cell, the liposome releases the Eno1 thereby allowing it to have its therapeutic effect.

d. Dendrimers

Dendrimers can be used as a scaffold for the attachment of multiple targeting moieties with one or more molecules of Eno1. In certain embodiments, the dendrimer is decorated with targeting moieties prior to coupling with Eno1.

Dendrimers can be used in the context of the invention as the backbone for targeted complexes for the delivery of non-intramuscularly administered Eno1 to muscle. Alternatively, dendrimers can be used to modulate the pharmacokinetic and pharmacodynamic properties of intramuscularly administered Eno1. In the compositions and methods of the invention, dendrimers are understood to be pharmaceutically acceptable dendrimers.

Dendrimer-based platforms have achieved attention for use in pharmaceutical applications Similar to other polymeric carriers, dendrimers can be synthesized to avoid structural toxicity and immunogenicity. The dendrimer's ability to mimic the size, solubility, and shape of human proteins makes the technology an ideal choice for many therapeutic and diagnostic applications. Being 1-10 nanometers in size enables dendrimers to efficiently diffuse across the vascular endothelium, internalize into cells, and be rapid cleared by the kidneys. This helps to avoid long-term toxicities and reduces the need for a rapidly degradable platform. The availability of multiple reactive surface groups enables the dendrimer to carry a higher payload of functional molecules, enhancing targeted delivery to the site of action, thereby increasing efficacy.

Dendrimers have been produced or are under commercial development for several biomedical applications. A topical, polylysine dendrimer-based microbicide, VivaGel™, has been developed by Starpharma. SuperFect® is a dendrimer-based material used for gene transfection. Dendrimer based diagnostic tools include Gadomer-17, a magnetic resonance imaging (MRI) contrast agent containing a polylysine dendrimer functionalized with gadolinium chelates, and Stratus® CS, a biosensor for cardiac markers to rapidly diagnosis heart attacks.

Dendrimers are defined by their core-shell structure, where the dendrimer approximately doubles in size and number of functional surface groups with each additional shell (or generation) added to the core. Shells are synthesized by alternating monomer reactions by means well known in the art. Specialized dendrimer backbones can be synthesized by varying the monomer units. The biological properties of the dendrimer are largely influenced by the chemical backbone and surface termination. For a dendrimer to be an appropriate vehicle for drug delivery in vivo, they must be non-toxic, non-immunogenic, and be capable of targeting and reaching specific locations by crossing the appropriate barriers while being stable enough to remain in circulation. The vast majority of the dendrimers synthesized and published in literature are insoluble in physiological conditions or are incapable of remaining soluble after the addition of functional molecules and are inappropriate for biological applications. However, several classes of dendrimers have been shown to be useful scaffolds for biomedical applications; examples include polyesters, polylysine, and polypropyleneimine (PPI or DAB) dendrimers.

The most widely used dendrimers in biomedical applications are poly(amidoamine) (PAMAM) dendrimers. The polyamide backbone synthesized from repeating reactions of methyl acrylate and ethylene-diamine helps the macromolecule maintain water solubility and minimizes immunogenicity. PAMAM dendrimers of different generation also are able to mimic the size and properties of globular proteins readily found in the body. The amine-terminated surface of full generation PAMAM dendrimers allows for easy surface modification, enabling the platform to carry and solubilize hydrophobic therapeutic molecules, such as methotrexate, in physiological conditions. PAMAM dendrimers exhibit little non-specific toxicity if the surface amines have been neutralized or appropriately modified (e.g., acylated).

Active targeting uses a molecule, such as targeting moiety, to mediate delivery of its payload (drug or otherwise) to cells by binding to cell-specific molecules. Targeting moieties, such as those provided herein, frequently bind through receptors highly expressed on target cells. The interactions between the targeting ligand and cell-surface receptor allow the therapeutic agent or payload to selectively reach muscle cells and even be ushered inside via receptor-mediated processes.

The multivalent effect associated with the display of multiple binding ligands on the dendrimer surface enhances the uptake of the dendritic scaffold compared to single ligands. Multivalent interactions, caused by the simultaneous binding of multiple ligands, allow for the dendrimers to increase the binding avidities of the platform, even when individual ligands have low affinities for the targeted receptor. The PAMAM platform has been successfully used as a scaffold for the attachment of multivalent targeting molecules including antibodies, peptides, T-antigens, and folic acid. The targeting ligands anchor the dendrimers to locations where specific receptors are expressed on cell surfaces. Targeted dendrimer-drug conjugates to deliver a higher dose specifically to targeted cells while avoiding normal cells, thus avoiding the potential systemic toxicity.

Neutralizing the surface amines of PAMAM dendrimers with acetyl groups minimizes toxicity and non-specific dendrimer uptake. The acetyl capping of the dendrimer also allows for increased clearance from the body, minimizing effects from long-term treatment. PEGylation of amino-terminated PAMAM dendrimers reduces immunogenicity and increases solubility. PEG terminated dendrimers have an increased half-life the blood stream as compared to the cationic parent material. Hydroxyl and methyoxyl terminated polyester dendrimers have been shown to be nontoxic in vivo up at concentrations up to 40 mg/kg. The differences in toxicities between cationic and anionic dendrimers have also been confirmed in vivo. Using a zebrafish embryo model, carboxyl terminated dendrimer was significantly less toxic than G4 amine-terminated dendrimer. In the same study, surface modification with RGD also reduced toxicity.

It will be understood that all of the dendrimers described above and herein may be used in the Eno1 compositions of the invention and their methods of use.

In certain embodiments, the ratio of the number of dendrimer molecules to the number of Eno1 molecules in the complex comprising dendrimer and Eno1 is between about 1:1 and about 10:1, e.g., about 1:1, about 2:1, about 3:1, about 4:1, about 5:1, about 6:1, about 7:1, about 8:1, about 9:1, or about 10:1. In one embodiment, the ratio of the number of dendrimer molecules to the number of Eno1 molecules in the complex comprising dendrimer and Eno1 is between about 3:1 and 7:1, e.g., 3:1, 4:1, 5:1, 6:1, or 7:1. In one embodiment, the ratio of the number of dendrimer molecules to the number of Eno1 molecules in the complex comprising dendrimer and Eno1 is between 4:1 and 6:1, e.g., 3:1, 4:1, or 5:1. In one embodiment, the ratio of the number of dendrimer molecules to the number of Eno1 molecules in the complex comprising dendrimer and Eno1 is between 3:1 and 5:1, e.g., 3:1, 4:1, or 5:1. In yet another embodiment, the ratio of the number of dendrimer molecules to the number of Eno1 molecules in the complex comprising dendrimer and Eno1 is between 4:1 and 5:1. In another embodiment, the ratio of the number of dendrimer molecules to the number of Eno1 molecules in the complex comprising dendrimer and Eno1 is between 3:1 and 4:1. In a further preferred embodiment, the ratio of the number of dendrimer molecules to the number of Eno1 molecules in the complex comprising dendrimer and Eno1 is about 5:1.

Optimal ratios of dendrimer to Eno1 in the complex may be tested and selected by assaying the Eno1 activity of the dendrimer/Eno1 complexes (e.g., as compared to uncomplexed Eno1) by using any routine methods known in the art, such as, for example, the pyruvate kinase (PK)/lactate dehydrogenase (LDH) assay or any other assays described herein. Optimal ratios of dendrimer to Eno1 may also be tested and selected by assessing the effect of the dendrimer/Eno1 complexes on glucose uptake in an in vitro assay, for example, by measuring glucose uptake in human skeletal muscle myotubes (HSMM) as described in Example 2 or any similar assays known in the art. Optimal ratios of dendrimer to Eno1 may also be tested and selected by measuring the effect of the dendrimer/Eno1 complexes on blood glucose levels in vivo, for example, by measuring the effect of the dendrimer/Eno1 complex on blood glucose in diabetic mouse models, as described in Examples 7 and 8 of US 2015/0361409, which is incorporated by reference herein in its entirety, or any similar models or assays known in the art. Optimal ratios of dendrimer to Eno1 in the complex will preferably retain Eno1 activity in vitro and/or in vivo, and/or provide delivery of Eno1 to cells.

It is understood that the compositions and methods of the invention include the administration of more than one, i.e., a population of dendrimer-Eno1-targeting peptide complexes. Therefore, it is understood that the number of dendrimer per Eno1 molecules can represent an average number of dendrimer per Eno1 in a population of complexes. In certain embodiments, at least 70% of the complexes have the selected molar ratio of dendrimer to Eno1. In certain embodiments, at least 75% of the complexes have the selected molar ratio of dendrimer to Eno1. In certain embodiments, at least 80% of the complexes have the selected molar ratio of dendrimer to Eno1. In certain embodiments, at least 85% of the complexes have the selected molar ratio of dendrimer to Eno1. In certain embodiments, at least 90% of the complexes have the selected molar ratio of dendrimer to Eno1.

In certain embodiments, the ratio of the number of dendrimer molecules to the number of targeting peptides in the dendrimer/Eno1/targeting peptide complex is between 1:0.1 and 1:10, between 1:1 and 1:10, between 1:1 and 1:5, or between 1:1 and 1:3. In certain embodiments the ratio of the number of dendrimer molecules to the number of targeting peptides is about 1:1, 1:2, 1:3, 1:4, 1:5, 1:6, 1:7, 1:8, 1:9 or 1:10. In a preferred embodiment, the ratio of the number of dendrimer molecules to the number of targeting peptides in the dendrimer/Eno1/targeting peptide complex is about 1:1. In a preferred embodiment, the ratio of the number of dendrimer molecules to the number of targeting peptides in the dendrimer/Eno1/targeting peptide complex is about 1:2. In a preferred embodiment, the ratio of the number of dendrimer molecules to the number of targeting peptides in the dendrimer/Eno1/targeting peptide complex is about 1:3.

In certain embodiments, the ratio of the number of targeting peptides to the number of dendrimer molecules in the dendrimer/Eno1/targeting peptide complex is at least 1:1, at least 2:1, at least 3:1, at least 4:1, at least 5:1, at least 6:1, at least 7:1, at least 8:1, at least 9:1 or at least 10:1. In one embodiment, the ratio of the number of targeting peptides to the number of dendrimer molecules in the dendrimer/Eno1/targeting peptide complex is at least 3:1.

It is understood that the compositions and methods of the invention include the administration of more than one, i.e., a population of targeting peptide-Eno1-dendrimer complexes. Therefore, it is understood that the number of targeting peptides per dendrimer can represent an average number of targeting peptides per dendrimer in a population of complexes. In certain embodiments, at least 70% of the complexes have the selected molar ratio of targeting peptides to dendrimer. In certain embodiments, at least 75% of the complexes have the selected molar ratio of targeting peptides to dendrimer. In certain embodiments, at least 80% of the complexes have the selected molar ratio of targeting peptide to dendrimer. In certain embodiments, at least 85% of the complexes have the selected molar ratio of targeting peptide to dendrimer. In certain embodiments, at least 90% of the complexes have the selected molar ratio of targeting peptide to dendrimer.

Optimal ratios of dendrimer to targeting peptide may be selected by measuring the targeting of the dendrimer/Eno1/targeting peptide complex to specific tissues in vivo, for example, by measuring the targeting of a detectably labeled dendrimer/Eno1/targeting peptide complex in vivo, as described in Example 6 of US 2015/0361409, which is incorporated by reference herein in its entirety.

e. Microparticles

Microparticles can be used as a scaffold for the attachment of multiple targeting moieties with one or more molecules of Eno1 either attached to or encapsulated in the microparticle. In certain embodiments, the microparticle is decorated with targeting moieties prior to coupling with Eno1.

f. Viral Vectors

Viral tropisms have long been studied and are used to direct viruses to the cell type of interest. Parker et al., 2013 (Gene Therapy, 20:1158-64) have developed an adenovirus serotype 5 capsite with the fiber and peton of serotype 35 to enhance delivery to skeletal and/or smooth muscle cells. Such viral vectors and other viral vectors can be used for the delivery of Eno1 expression constructs to muscle cells.

### V. FORMULATIONS, DOSAGES AND MODES OF ADMINISTRATION

Techniques and dosages for administration vary depending on the type of compound (e.g., protein and/or nucleic acid, alone or complexed with a microparticle, liposome, or dendrimer) and are well known to those skilled in the art or are readily determined.

Therapeutic compounds of the present invention may be administered with a pharmaceutically acceptable diluent, carrier, or excipient, in unit dosage form. Administration may be parenteral, intravenous, subcutaneous, oral, topical, or local. In certain embodiments, administration is not oral. In certain embodiments, administration is not topical. In certain preferred embodiments, administration is systemic. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, subcutaneous delivery, etc.

The composition can be in the form of a pill, tablet, capsule, liquid, or sustained release tablet for oral administration; or a liquid for intravenous, subcutaneous, or parenteral administration; or a polymer or other sustained release vehicle for systemic administration.

Methods well known in the art for making formulations are found, for example, in “Remington: The Science and Practice of Pharmacy” (20th ed., ed. A. R. Gennaro, 2000, Lippincott Williams & Wilkins, Philadelphia, Pa.). Formulations for parenteral administration may, for example, contain excipients, sterile water, saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Nanoparticulate formulations (e.g., biodegradable nanoparticles, solid lipid nanoparticles, liposomes) may be used to control the biodistribution of the compounds. Other potentially useful parenteral delivery systems include ethylene-vinyl acetate copolymer particles, osmotic pumps, implantable infusion systems, and liposomes. The concentration of the compound in the formulation varies depending upon a number of factors, including the dosage of the drug to be administered, and the route of administration.

The compound may be optionally administered as a pharmaceutically acceptable salt, such as non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids and the like; polymeric acids such as tannic acid, carboxymethyl cellulose, and the like; and inorganic acid such as hydrochloric acid, hydrobromic acid, sulfuric acid phosphoric acid, and the like. Metal complexes include zinc, iron, and the like.

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and anti-adhesives (e.g., magnesium stearate, zinc stearate, stearic acid, silicas, hydrogenated vegetable oils, or talc). Formulations for oral use may also be provided as chewable tablets, or as hard gelatin capsules wherein the active ingredient is mixed with an inert solid diluent, or as soft gelatin capsules wherein the active ingredient is mixed with water or an oil medium.

The dosage and the timing of administering the compound depend on various clinical factors including the overall health of the subject and the severity of the symptoms of disease, e.g., diabetes, pre-diabetes.

### 1. Formulations for Long Acting Injectable Drugs

Biologics and other agents subject to high rates of first pass clearance may not be amenable to oral administration and require administration by parenteral routes. However, compliance with treatment regimens for injectable drugs can be low as subjects are often adverse to self-administering agents by injection, e.g., subcutaneous injection, particularly when the disease does not make the subject feel sick. Other routes of administration by injection, e.g., intravenous, intramuscular, typically require administration by a trained professional, making frequent administration of the agent inconvenient and often painful.

Formulations have been created to provide sustained delivery of injectable agents including, but not limited to, oil-based injections, injectable drug suspensions, injectable microspheres, and injectable in situ systems. Long-acting injectable formulations offer many advantages when compared with conventional formulations of the same compounds. These advantages include, at least, the following: a predictable drug-release profile during a defined period of time following each injection; better patient compliance; ease of application; improved systemic availability by avoidance of first-pass metabolism; reduced dosing frequency (i.e., fewer injections) without compromising the effectiveness of the treatment; decreased incidence of side effects; and overall cost reduction of medical care.

a. Oil-Based Injectable Solutions and Injectable Drug Suspensions.

Conventional long-acting injections consist either of lipophilic drugs in aqueous solvents as suspensions or of lipophilic drugs dissolved in vegetable oils. Commercially available oil based injectable drugs for intramuscular administration include, but are not limited to, haloperidol deconate, fluphenazine deconate, testosterone enanthate, and estradiol valerate. Administration frequency for these long-acting formulations is every few weeks or so. In the suspension formulations, the rate-limiting step of drug absorption is the dissolution of drug particles in the formulation or in the tissue fluid surrounding the drug formulation. Poorly water-soluble salt formations can be used to control the dissolution rate of drug particles to prolong the absorption. However, several other factors such as injection site, injection volume, the extent of spreading of the depot at the injection site, and the absorption and distribution of the oil vehicle per se can affect the overall pharmacokinetic profile of the drug. Modulation of these factors to provide the desired drug release profile is within the ability of those of skill in the art.

b. Polymer-Based Microspheres and In-Situ Formings.

The development of polymer-based long-acting injectables is one of the most suitable strategies for macromolecules such as peptide and protein drugs. Commercially available microsphere preparations include, but are not limited to, leuprolide acetate, triptorelin pamoate, octreotide acetate, lanreotide acetate, risperidone, and naltrexone. Commercially available in situ forming implants include leuprolide acetate, and in situ forming implants containing paclitaxel and bupivacaine are in clinical trials. These formulations are for intramuscular administration. Advantages of polymer-based formulations for macromolecules include: in vitro and in vivo stabilization of macromolecules, improvement of systemic availability, extension of biological half life, enhancement of patient convenience and compliance, and reduction of dosing frequency.

The most crucial factor in the design of injectable microspheres and in situ formings is the choice of an appropriate biodegradable polymer. The release of the drug molecule from biodegradable microspheres is controlled by diffusion through the polymer matrix and polymer degradation. The nature of the polymer, such as composition of copolymer ratios, polymer crystallinities, glass-transition temperature, and hydrophilicities plays a critical role in the release process. Although the structure, intrinsic polymer properties, core solubility, polymer hydrophilicity, and polymer molecular weight influence the drug-release kinetics, the possible mechanisms of drug release from microsphere are as follows: initial release from the surface, release through the pores, diffusion through the intact polymer barrier, diffusion through a water-swollen barrier, polymer erosion, and bulk degradation. All these mechanisms together play a part in the release process. Polymers for use in microsphere and in situ formings include, but are not limited to a variety of biodegradable polymers for controlled drug delivery intensively studied over the past several decades include polylactides (PLA), polyglycolides (PGA), poly(lactide-co-glycolide) (PLGA), poly(ε-caprolactone) (PCL), polyglyconate, polyanhydrides, polyorthoesters, poly(dioxanone), and polyalkylcyanoacrylates. Thermally induced gelling systems used in in situ formings show thermo-reversible sol/gel transitions and are characterized by a lower critical solution temperature. They are liquid at room temperature and produce a gel at and above the lower critical solution temperature. In situ solidifying organogels are composed of water-insoluble amphiphilic lipids, which swell in water and form various types of lyotropic liquid crystals.

### VI. METHODS OF TREATMENT

As demonstrated herein, administration of Eno1 protein reduces rosiglitazone-induced weight gain in a diabetic mouse model. Accordingly, the present invention provides, in one aspect, a method of treating obesity in a subject in need thereof, comprising administering to the subject a therapeutically effective amount of a composition comprising Eno1 or a fragment thereof, thereby treating obesity in the subject.

In one embodiment the subject is obese or suffering from obesity, i.e. has a body mass index (BMI) equal to or greater than 30 kg/m². In some embodiments the subject is obese and is afflicted with diabetes, e.g. type 2 diabetes, type 1 diabetes, or pre-diabetes. In some embodiments, the subject is obese, afflicted with diabetes, and the obesity condition is exacerbated by a therapeutic treatment. In some embodiments, the therapeutic treatment is administration of a drug that induces weight gain. In some embodiments, the drug that induces weight gain is a drug for treatment of diabetes. In a particular embodiment, the diabetic drug is rosiglitazone.

In some embodiments, the subject is obese and is not afflicted with diabetes. For example, in some embodiments, the subject is not afflicted with diabetes and the obesity condition is caused or exacerbated by a therapeutic treatment, for example, administration of a drug that induces weight gain. In some embodiments, the drug that induces weight gain is not a drug for treatment of diabetes, e.g., the diabetic drug is not rosiglitazone.

In another aspect, the present invention provides a method of reducing body weight in a subject comprising administering to the subject a therapeutically effective amount of a composition comprising Eno1 or a fragment thereof, thereby reducing body weight in the subject.

In some embodiments the subject is obese, i.e., has a body mass index (BMI) equal to or greater than 30 kg/m². In some embodiments, the subject in not obese, but is at risk of becoming obese. For example, in some embodiments the subject is overweight, i.e. has a body mass index (BMI) greater than or equal to 25 kg/m² and less than 30 kg/m². In some embodiments the subject is obese or overweight and is afflicted with diabetes, e.g. type 2 diabetes, type 1 diabetes, or pre-diabetes. In some embodiments, the subject is obese or overweight, afflicted with diabetes, and the obesity or overweight condition is exacerbated by a therapeutic treatment. In some embodiments, the therapeutic treatment is administration of a drug that induces weight gain. In some embodiments, the drug that induces weight gain is a drug for treatment of diabetes. In a particular embodiment, the diabetic drug is rosiglitazone.

In some embodiments, the subject is obese or overweight and is not afflicted with diabetes. For example, in some embodiments, the subject is not afflicted with diabetes and the obesity or overweight condition is caused or exacerbated by a therapeutic treatment, for example, administration of a drug that induces weight gain. In some embodiments, the drug that induces weight gain is not a drug for treatment of diabetes, e.g., the diabetic drug is not rosiglitazone.

In another aspect, the invention provides a method of reducing or preventing body weight gain in a subject, comprising administering to the subject a therapeutically effective amount of a composition comprising Eno1 or a fragment thereof, thereby reducing or preventing body weight gain in the subject.

In various embodiments, the composition is administered to a subject in need of reducing or preventing body weight gain. For example, in certain embodiments the subject is at risk or increased risk for gaining body weight. For example, in certain embodiments the subject is in need of receiving a therapeutic treatment, e.g., administration of an active agent or drug, that induces, is known to induce, or has the capacity to cause weight gain. Therapeutic agents known to induce or have the capacity to cause weight gain would be recognized by one of skill in the art. For example, in some embodiments, the subject is in need of treatment with a therapeutic treatment that induces or has the capacity to cause weight gain, wherein the therapeutic treatment is selected from the group consisting of an anti-psychotic agent, an antidepressant, a mood stabilizer, an anticonvulsant, a steroid hormone, a beta-blocker, an oral contraceptive, an antihistamine, an HIV antiretroviral drug, an antihyperlipemic agents, a hypotensive or antihypertensive agent, a chemotherapeutic agent, an immunotherapeutic agent, and an immunosuppressive agent. In some embodiments, the subject is in need of treatment with a therapeutic treatment that induces or has the capacity to cause weight gain, wherein the therapeutic treatment is a diabetic drug. In other embodiments, the subject is at risk for weight gain due to changes in hormone levels, such as during premenopause or menopause in women, or due to hypothyroidism, cushing syndrome or increased cortisol (stress hormone) production. In other embodiments, the subject is at risk for weight gain because the subject is suffering from polycystic ovarian syndrome (PCOS).

In some embodiments, the subject is afflicted with a disorder selected from the group consisting of psychosis, depression, HIV, hypertension, cancer and an immune disorder. In some embodiments, the subject has any one or more of elevated blood glucose, decreased glucose tolerance, decreased insulin sensitivity and/or insulin resistance, diabetes, elevated Hb1Ac level, and abnormal blood glucose level control. In some embodiments, the subject is obese or overweight, and is at risk for further body weight gain due to any of the factors described herein.

The methods described above may further comprise selecting a patient for treatment with the composition comprising Eno1 or a fragment thereof. For example, in some embodiments, the methods further comprise selecting a subject having any one or more of obesity, overweight, elevated blood glucose, decreased glucose tolerance, decreased insulin sensitivity and/or insulin resistance, diabetes, elevated Hb1Ac level, and abnormal blood glucose level control. In some embodiments the methods further comprise selecting a subject afflicted with a disorder selected from the group consisting of psychosis, depression, HIV, hypertension, cancer and an immune disorder. In some embodiments, the methods further comprise selecting a subject at risk for weight gain. In some embodiments the methods comprise selecting a subject in need of treatment for a disorder selected from the group consisting of psychosis, depression, HIV, hypertension, cancer and an immune disorder. In some embodiments the methods further comprise selecting a subject in need of treatment for, or who is undergoing treatment for, a disorder selected from the group consisting of psychosis, depression, HIV, hypertension, cancer and an immune disorder, wherein the treatment causes or induces weight gain.

In certain embodiments, the administration of Eno1 to a subject reduces body weight in the subject relative to a control, or reduces or prevents body weight gain in the subject relative to a control. In some embodiments, the control is one or more control subjects that has not been administered Eno1. In some embodiments, the control is an average from a group or population of subjects that have not been administered Eno1, e.g., a predetermined average from said group or population. In some embodiments, the control subject has a similar clinical situation as the subject being administered Eno1. For example, in some embodiments, the subject is administered Eno1 in combination with a diabetic drug, while the control subject is administered the same diabetic drug but is not administered Eno1.

In certain embodiments of the invention, administration of Eno1 and optionally one or more additional therapeutic agents results in a reduction in BMI of at least 1%, 2%, 3%, 4%, 5%, 6%, 7%, 8%, 9%, 10%, 15%, 20%, 25%, 30%, 40%, 50%, 60%, 70% or 80% relative to a control, e.g. a subject or a population of subjects that has not been administered Eno1. In certain embodiments, administration of Eno1 and optionally one or more additional therapeutic agents results in a reduction in body weight of at least 1%, 2%, 3%, 4%, 5%, 6%, 7%, 8%, 9%, 10%, 15%, 20%, 25%, 30%, 40%, 50%, 60%, 70% or 80% relative to a control, e.g. a subject or a population of subjects that has not been administered Eno1. In certain embodiments, administration of Eno1 attenuates body weight gain by at least 1%, 2%, 3%, 4%, 5%, 6%, 7%, 8%, 9%, 10%, 15%, 20%, 25%, 30%, 40%, 50%, 60%, 70% or 80%, relative to a control, e.g. a subject or a population of subjects that has not been administered Eno1.

In certain embodiments, the subject that is administered Eno1 and optionally one or more additional therapeutic agents has a BMI of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, or 130 kg/m². Any of these values may be used to define a range for the BMI of a subject. For example the BMI of a subject may range from 25-30 kg/m², 30-40 kg/m², or 30-100 kg/m². In certain embodiments, the subject that is administered Eno1 and optionally one or more additional therapeutic agents has a BMI of at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100 kg/m².

### 1. Combination Therapies

In one embodiment of the methods of the invention, the method further comprises administering an additional therapeutic agent, e.g., diabetes mellitus-treating agents, diabetic complication-treating agents, antihyperlipemic agents, hypotensive or antihypertensive agents, anti-obesity agents, diuretics, chemotherapeutic agents, immunotherapeutic agents and immunosuppressive agents. Eno1 and the additional therapeutic agent may act additively or synergistically. In one embodiment, Eno1 is administered concurrently with the administration of the additional therapeutic agent. In another embodiment, Eno1 is administered prior or subsequent to administration of the additional therapeutic agent.

For example, the methods of treatment of obesity, reducing body weight and preventing body weight gain using Eno1 as described herein can be combined with known methods and agents for the treatment of diabetes. Many agents and regimens are currently available for treatment of diabetes. The specific agent selected for treatment depends upon the subject, the specific symptoms and the severity of the disease state. For example, in certain embodiments, Eno1 can be administered in conjunction with dietary and/or behavior modification, e.g., caloric restriction, alone or in combination with bariatric surgery, and/or with increased physical activity. In certain embodiments, Eno1 can be administered with a diabetic drug, e.g. a drug for treatment of type 2 diabetes. Drugs for treatment of type 2 diabetes include, but are not limited to, GLP-1 (glucagon-like peptide 1) receptor agonists (e.g. GLP-1 peptide, incretin mimetics, exenatide (Byetta/Bydureon), liraglutide (Victoza, Saxenda), lixisenatide (Lyxumia), albiglutide (Tanzeum), dulaglutide (Trulicity)); meglitinides (repaglinide (Prandin/Prandimet) and nateglinide (Starlix); sulfonylureas (glipizide (Glucotrol/Metaglip), glimepiride (Amaryl/Duetact/Avandaryl), glyburide (DiaBeta, Glynase, Micronase, Glucovance), gliclazine, chloropropamide (Diabinese, tolazamide (Tolinase), and tolbutamide (Orinase, Tol-Tab)); Dipeptidy peptidase-4 (DPP-4) inhibitors (saxagliptin (Onglyza/Kombiglyze), sitagliptin (Januvia/Janumet/Juvisync), alogliptin (Nesina/Kazano/Oseni), linagliptin (Tradjenta/Glyxambi/Jentadueto)); biguanides (metformin (Fortamet, Glucophage, Riomet, Glumetza, Metformin Hydrochloride ER)); thiazolidinediones (rosiglitazone (Avandia/Avandaryl/Amaryl M) and pioglitazone (Actos/Oseni/Actoplus)); amylinomimetic drugs (pramlintide (Symlin)); dopamine agonists (bromocriptine (Parlodel, Cyclo set)); sodium glucose transporter 2 (SGLT-2) inhibitors (dapagliflozin (Farxiga/Xigduo XR), canagliflozin (Ivokana/Ivokamet), empagliflozin (Jardiance/Glyxambi/Synjardy), ipraglifozin, tofogliflozin, luseoglifozin, ertugliflozin, LX 4211, EGT001442, GW 869682, and ISIS 388626); bile acid sequestrants (colesevelam hydrochloride (Welchol)); and alpha-glucosidase inhibitors (acarbose (Precose) and miglitol (Glyset)). Insulins are typically used only in treatment of later stage type 2 diabetes and include rapid-acting insulin (insulin aspart (NovoLog), insulin glulisine (Apidra), insulin lispro (Humalog), insulin inhalation powder (Afrezza)); short-acting insulin (insulin regular (Humulin R, Novolin R)); intermediate-acting insulin (insulin NPH human (Humulin N, Novolin N)), and long-acting insulin (insulin glargine (Lantus, Toujeo), insulin detemir (Levemir), and insulin degludec (Tresiba)). Agents for the treatment of diabetes are known in the art and are described, for example, in Cherney, 2016, A Complete List of Diabetes Medications, Healthline, retrieved from healthline.com/health/diabetes/medications-list; and Chao, 2014, Clinical Diabetes 32(1): 4-11, each of which is incorporated herein in its entirety. Treatments for diabetes can also include behavior modification including exercise and weight loss which can be facilitated by the use of drugs or surgery. Treatments for elevated blood glucose and diabetes can be combined. For example, drug therapy can be combined with behavior modification therapy.

In certain embodiments, Eno1 is administered with a therapeutic agent that induces weight gain in a subject. In certain embodiment, the therapeutic agent that induces weight gain is a diabetic drug. Therapeutic agents for the treatment of diabetes that induce weight gain include, but are not limited to, sulfonylureas, insulin, GLP-1 receptor agonists, DPP-4 inhibitors, metformin, rosiglitazone, pioglitazone, glyburide repaglinide and tolbutamide. In a further particular embodiment, Eno1 is administered with a GLP-1 receptor agonist and a DPP-4 inhibitor.

In certain embodiments, the therapeutic agent that induces weight gain is an antipsychotic agent. Antipsychotic agents that induce weight gain include, but are not limited to, amisulpride, aripiprazole, asenapine, blonanserin, bifeprunox, clotiapine, clozapine, iloperidone, lithium, lurasidone, mosapramine, melperone, olanzapine, paliperidone, perospirone, pimavanserin, quepin, quetiapine, remoxipride, risperidone, sertindole, sulpiride, vabicaserin, ziprasidone, and zotepine. Antipsychotic agents that induce weight gain are described for example in Vieweg et al. (2012, Focal Point: Youth, Young Adults, & Mental Health. Healthy Body—Healthy Mind, Summer, 26(1): 19-22) and US 2014/0349999, each of which is incorporated by reference herein in its entirety.

Additional therapeutic agents that induce weight gain in a subject include, but are not limited to antidepressants (e.g., citalopram (Celexa), fluoxetine (Prozac), fluvoxamine (Luvox), paroxetine (Paxil), and sertraline (Zoloft)), mood stabilizers, anticonvulsants, steroid hormones (e.g., methylprednisolone (Medrol), prednisolone (Orapred, Pediapred, Prelone), prednisone (Deltasone, Prednicot, and Sterapred), beta-blockers (e.g., acebutolol (Sectral), atenolol (Tenormin), metoprolol (Lopressor, Toprol XL), and propranolol (Inderal), oral contraceptives, antihistamines (e.g., cetirizine (Zyrtec), diphenhydramine (Benadryl), fexofenadine (Allegra), and loratadine (Claritin), HIV antiretroviral drugs, antiseizure and antimigraine drugs (e.g., amitriptyline (Elavil), nortriptyline (Aventyl, Pamelor), and valproic acid (Depacon, Depakote, Stavzor), and protease inhibitors. See 2010/0215635, which is incorporated by refrence herein in its entirety. Therapeutic agents that induce weight gain are described, for example, in Booth, 2015, Are Your Meds Making you Gain Weight?, WebMD, retrieved from webmd.com/diet/obesity/medication-weight-gain, which is incorporated herein in its entirety.

Examples of other therapeutic agents which can be used with Eno1 include, but are not limited to, diabetic complication-treating agents, antihyperlipemic agents, hypotensive or antihypertensive agents, anti-obesity agents, diuretics, chemotherapeutic agents, immunotherapeutic agents, immunosuppressive agents, and the like.

Examples of agents for treating diabetic complications include, but are not limited to, aldose reductase inhibitors (e.g., tolrestat, epalrestat, zenarestat, zopolrestat, minalrestat, fidareatat, SK-860, CT-112 and the like), neurotrophic factors (e.g., NGF, NT-3, BDNF and the like), PKC inhibitors (e.g., LY-333531 and the like), advanced glycation end-product (AGE) inhibitors (e.g., ALT946, pimagedine, pyradoxamine, phenacylthiazolium bromide (ALT766) and the like), active oxygen quenching agents (e.g., thioctic acid or derivative thereof, a bioflavonoid including flavones, isoflavones, flavonones, procyanidins, anthocyanidins, pycnogenol, lutein, lycopene, vitamins E, coenzymes Q, and the like), cerebrovascular dilating agents (e.g., tiapride, mexiletene and the like).

Antihyperlipemic agents include, for example, statin-based compounds which are cholesterol synthesis inhibitors (e.g., pravastatin, simvastatin, lovastatin, atorvastatin, fluvastatin, rosuvastatin and the like), squalene synthetase inhibitors or fibrate compounds having a triglyceride-lowering effect (e.g., fenofibrate, gemfibrozil, bezafibrate, clofibrate, sinfibrate, clinofibrate and the like).

Hypotensive agents include, for example, angiotensin converting enzyme inhibitors (e.g., captopril, enalapril, delapril, benazepril, cilazapril, enalapril, enalaprilat, fosinopril, lisinopril, moexipril, perindopril, quinapril, ramipril, trandolapril and the like) or angiotensin II antagonists (e.g., losartan, candesartan cilexetil, olmesartan medoxomil, eprosartan, valsartan, telmisartan, irbesartan, tasosartan, pomisartan, ripisartan forasartan, and the like).

Antiobesity agents include, for example, central antiobesity agents (e.g., dexfenfluramine, fenfluramine, phentermine, sibutramine, amfepramone, dexamphetamine, mazindol, phenylpropanolamine, clobenzorex and the like), gastrointestinal lipase inhibitors (e.g., orlistat and the like), β-3 agonists (e.g., CL-316243, SR-58611-A, UL-TG-307, SB-226552, AJ-9677, BMS-196085 and the like), peptide-based appetite-suppressing agents (e.g., leptin, CNTF and the like), cholecystokinin agonists (e.g., lintitript, FPL-15849 and the like), serotonin 2C receptor agonists (e.g., lorcaserin (Belviq)), monoamine reuptake inhibitors (e.g., tesofensine), and the like. Antiobesity agents can also include drug combinations, including combinations of opiod antagonists (naltrexone) and antidepressants (buproprion), such as Contrave; combinations of phentermine and antiseizure agents (topiramate), such as Qsymia; combinations of antidepressants (buproprion) and antiseizure agents (zonsiamide), such as Empatic. See Adan, 2013, Trends Neurosci., 36(2): 133-40; Gustafson et al., 2013, P. T., 38(9): 525-34; Shin and Gadde, 2013, Diabetes Metab. Syndr. Obes., 6: 131-9; Bello and Zahner, 2009, Curr. Opin. Investig. Drugs, 10(10) 1105-16, each of which is incorporated herein in its entirety.

This invention is further illustrated by the following examples which should not be construed as limiting. The contents of all references and published patents and patent applications cited throughout the application are hereby incorporated by reference.

### EXAMPLES

### Example 1

### Reduction of Weight Gain by Treatment with Muscle Targeted Eno1/Dendrimer Complex and Rosiglitazone in a Genetic Model of Obesity, Db/Db Mice

A muscle targeted Eno1/dendrimer complex was generated to analyze its efficacy in reducing weight gain. The dendrimer complex comprised human Eno1, transcript variant 1 protein (SEQ ID NO: 2) which was non-covalently linked to a G5-PAMAM dendrimer/muscle targeting peptide (MTP) (ASSLNIA; SEQ ID NO: 7) conjugate. Stock solutions of Eno1 were prepared in buffer and the protein solution was mixed with the G5 dendrimer-MTP conjugate.

Lean mice and male obese and diabetic db/db mice (male BKS.Cg-m+/+Lepr^{db}/J) mice were obtained from a commercial vendor. All mice were housed 2-3 per cage at 22° C. on a 12:12 hr day-night cycle and were acclimated for 3 weeks at animal facility on a standard chow diet. At 8 weeks of age, 200 μg/kg body weight Eno1 was administered twice daily (at 9:00 a.m. and 5:00 p.m., 400 μg/kg daily dose) by subcutaneous injection, and 20 mg/kg body weight rosiglitazone was administered once daily by gavage at 9:00 a.m. Lean mice and db/db mice also received subcutaneous injections of saline as a control. The treatment groups were as follows:
1. lean mice with saline injection (control)
2. db/db mice with saline injection (control)
3. db/db mice with rosiglitazone (20 mg/kg, once daily)
4. db/db mice with rosiglitazone (20 mg/kg, once daily)+Eno1 (200 μg/kg, twice daily)

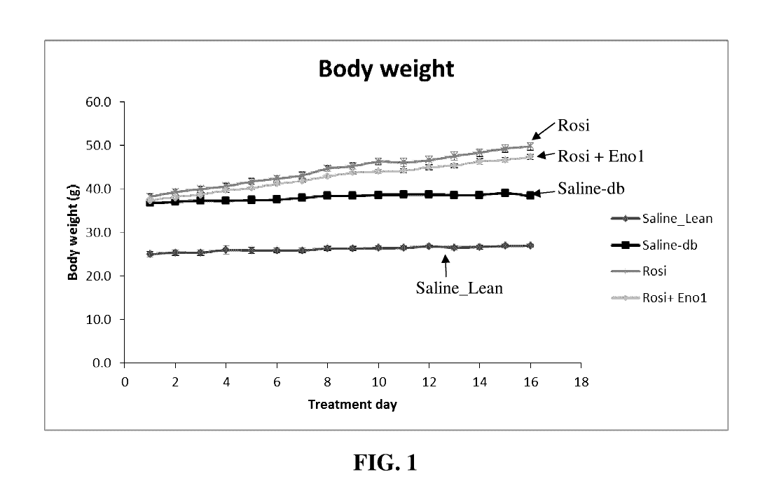

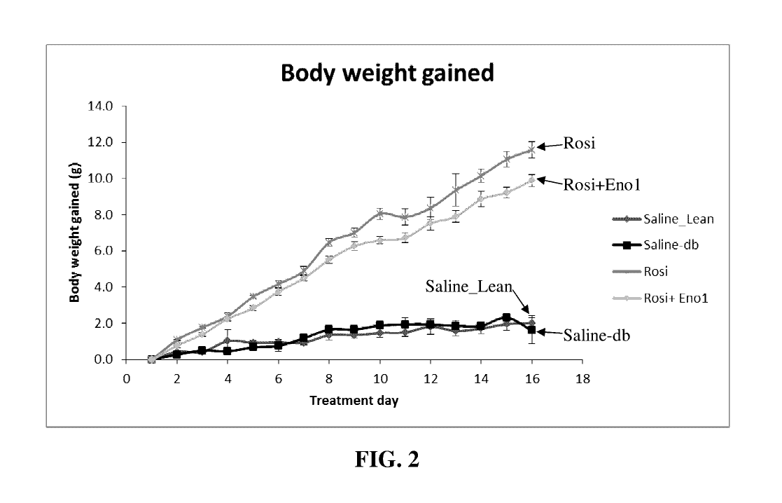

The mice were weighed daily to determine the effect of rosiglitazone and Eno1 on body weight gain. As shown in FIGS. 1 and 2, rosiglitazone alone and rosiglitazone+Eno1 showed increased body weight compared to control (saline treated) db/db mice. However, body weight was lower in the rosiglitazone+Eno1 treatment group compared to rosiglitazone alone, indicating that Eno1 attenuates rosiglitazone-induced weight gain.

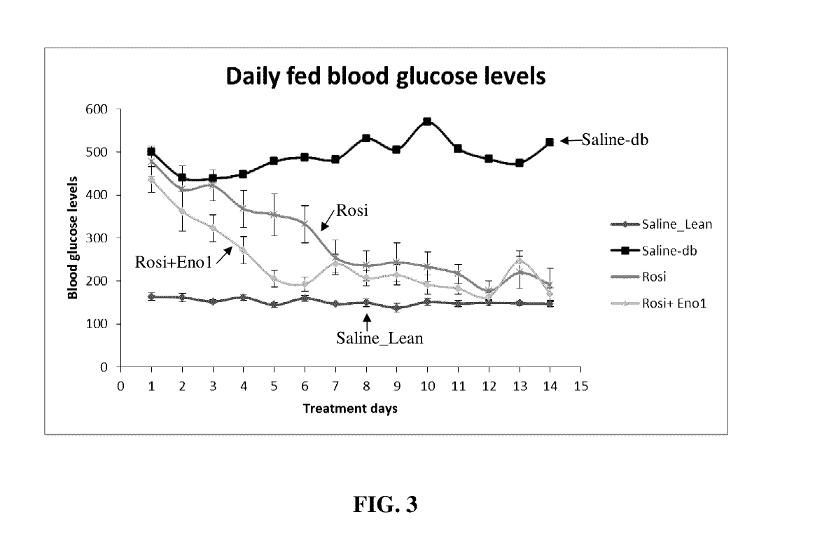

The effect of Eno1 on lowering fed blood glucose was also tested in the db/db mice. Specifically, without controlling the intake of food, blood glucose levels in mice were measured once per day in the morning immediately before Eno1 and/or rosiglitazone treatment. The combination of rosiglitazone and Eno1 reduced blood glucose levels more quickly than rosiglitazone alone (FIG. 3).

While not wishing to be bound by theory, it is likely that muscle-targeted Eno1 limits glucose mediated fat storage in adipose tissue typically induced by rosiglitazone treatment by diverting some glucose to skeletal muscle for utilization (i.e. oxidation).

### Example 2

### Generation of a Detectably Labeled PAMAM Dendrimer, Muscle Targeted Eno1

A detectably labeled muscle targeted Eno1 was generated to analyze its efficacy in targeting to muscle cells. Detectably labeled G5-PAMAM dendrimers containing the muscle targeting peptide (MTP) ASSLNIA and/or Eno1 were generated using the methods described below. A range of different ratios of MTP to dendrimer were evaluated, including MTP containing dendrimers which contained about 10 MTP peptides per dendrimer, about 3 MTP peptides per dendrimer, or about 1 MTP peptide per dendrimer.

The process of preparing Eno1 dendrimer complexes includes the identification of optimal ratios and concentrations of the reagents. Stock solutions of Eno1 were prepared in buffer and the protein solution was mixed with G5 dendrimer-muscle targeting peptide (MTP) conjugate in different ratios. A range of different ratios of dendrimer to Eno1 were also evaluated, including Eno1 containing dendrimers which contained about one dendrimer per molecule of Eno1 protein or about five dendrimers per molecule of Eno1 protein.

The stability of the Eno1-dendrimer-SMTP complex was evaluated at different temperatures, and stability was determined over a 3-4 month time period by measuring Eno1 activity using a commercially available Eno1 assay. The selected conjugates were also evaluated using biophysical techniques, including Dynamic Light Scattering (DLS) and UV-Vis spectroscopy to confirm complexation between the dendrimer-peptide conjugate and Eno1.

Determination of the Purity of Eno1:

The purity of a 5.32 mg/mL solution of Eno1 protein was checked by Coomassie and Silver staining and Western blotting. Several dilutions of the Eno1 protein ranging from 10 μg/well to 100 ng/well were prepared and loaded on a 12-well, 4-12% mini-PROTEAN® TGX gel [BIO-RAD Cat#456-1095 Lot#4000 79200]. The lane assignments were as follows; Lane 1: Ladder (Precision Plus Protein Standard Dual Color [BIO-RAD Cat#161-0374]; Lane 2: Eno1 (10.0 μg); Lane 3: Eno1 (1.0 μg); Lane 4: Eno1 (0.1 μg); Lane 5: Ladder (Precision Plus Protein Standard Dual Color [BIO-RAD Cat#161-0374]; Lane 6: Eno1 (10.0 μg); Lane 7: Eno1 (1.0 μg); Lane 8: Eno1 (0.1 μg); Lane 9: Ladder (Precision Plus Protein Standard Dual Color [BIO-RAD Cat#161-0374]; Lane 10: Eno1 (10.0 μg); Lane 11: Eno1 (1.0 μg); Lane 12: Eno1 (0.1 μg). The SDS-PAGE was run at 200 V for 20-25 min.

Coomassie Staining:

After the gel was run, the gel was split into 3 equal parts. One of the parts was stained with Coomassie Stain. Briefly, the gel was soaked in 100 mL of Coomassie Stain solution (0.025% Coomassie Stain in 40% Methanol and 7% Acetic Acid) and heated for one minute in a microwave. Then the gel was left to stain with gentle agitation for 45 minutes. After the staining was complete, the gel was destained using destaining solution (40% Methanol and 7% Acetic Acid) until the background staining was acceptable. The protein ran as a single band of about 47 KDa, which is consistent with the size of Eno1.

Silver Staining:

Since Coomassie Staining is not a sensitive method for visualization of the protein bands, another portion of the gel was stained with Silver Stain using BIO-RAD's Silver Staining Kit [BIO-RAD Cat#161-0443]. The Modified Silver Stain Protocol was followed. Coomassie staining indicated that overall purity of the Eno1 was relatively high.

Western Blot Analysis:

The identity of Eno1 was further confirmed by Western blot.

For this purpose, the final portion of the gel was transferred into 100 mL of Tris-Glycine buffer and transferred onto 0.2 μm PVDF membrane (BIO-RAD) using a transblot SD semi-dry transfer apparatus (BIO-RAD) at 20 V for 2.0 h. The efficiency of the transfer was checked by observing the presence of the pre-stained ladder bands on the membrane. The membrane was dried for 1.0 h. The membrane was then wetted with methanol for 1.0 min and blocked with 15.0 mL ODYSSEY® Blocking Buffer (LICOR) at room temperature for 2.0 h.

After the blocking was complete, the membrane was incubated with 15.0 mL ODYSSEY® Blocking Buffer containing 30 μL of anti-ENOA-1 m-Ab (mouse) (purchased from ABNOVA) overnight at 4° C. Then the membrane was washed with 3×30 mL of 1×PBS-T with shaking for 5 minutes each. The membrane was incubated with 15.0 mL ODYSSEY® Blocking Buffer containing 5 μL of Goat anti-mouse secondary antibody labeled with IRDye® 800CW (purchased from LICOR) for 2.0 h at room temperature. After the incubation, the membrane was washed with 3×30 mL of 1×PBS-T followed by 2×30 mL of 1×PBS with shaking for 5 minutes each. Finally, the membrane was imaged using the LICOR ODYSSEY Infrared Imager. Western Blot analysis confirmed that the dominant band at 47 kDa was Eno1.

Zeta (ζ)-Potential Characterization of Enolase-I/G5-PAMAM-SMTP:

Eno1 and Generation 5 PAMAM dendrimers decorated with 2-3 Skeletal Muscle Targeting Peptides (SMTPs) were complexed at varied ratios to form Eno1/G5-SMTP protein/dendrimer complexes. The concentration of the dendrimer was kept constant at 1.0 μM and the Eno1 concentration was varied between 0.1 μM-10.0 μM. Table 2 below describes how the Enolase-I/G5-dendrimer/SMTP mixtures were prepared.

TABLE 2
Various combinations of Eno1 and G5-dendrimer/SMTP for formation of
dendrimer complexes.

| | | G5-Dendrimer | |
|---|---|---|---|
| Eno1/Dendrimer | Eno1 | SMTP | PBS buffer |
| Molar Ratio | (5.32 mg/mL) | (30.0 mg/mL) | pH = 7.40 |
| 10:1 | 88.3 μL | 1.03 μL | 910.67 μL |
| 5:1 | 44.15 μL | 1.03 μL | 954.82 μL |
| 2:1 | 17.66 μL | 1.03 μL | 981.31 μL |
| 1:1 | 8.83 μL | 1.03 μL | 990.14 μL |
| 1:2 | 4.42 μL | 1.03 μL | 994.55 μL |
| 1:5 | 1.77 μL | 1.03 μL | 997.2 μL |
| 1:10 | 0.88 μL | 1.03 μL | 998.09 μL |

Each sample was prepared by adding G5-dendrimer/SMTP to the respective amount of PBS. Enolase was then added to the G5-dendrimer/SMTP solution in a drop wise fashion while vortexing at low speed. The sample was then incubated at room temperature for 20 minutes prior to analysis.

Size measurements were made using the Zetasizer Nano Z90s instrument from Malvern Instruments. The default parameters were used for the measurements and three separate measurements of each sample were collected. Zeta (ζ)-Potential data for three samples of Eno1/G5-dendrimer/SMTP complexes having a 2:1 molar ratio of Eno1 to dendrimer/SMTP were collected. Zeta (ζ)-Potential was measured using Dynamic Light Scattering. The peaks of the three samples matched, indicating a uniform charge distribution of the Enolase-SMTP dendrimer complex.

Stability of Enolase-I/G5-SMTP Complexes:

The stability of the Enolase-I/G5-dendrimer/SMTP conjugates was measured by using the ENO1 Human Activity Assay Kit (ABCAM, Cambridge, Mass.; Catalogue No. ab117994). Briefly, the sample was added to a microplate containing a monoclonal mouse antibody specific to Eno1. The microplate was incubated at room temperature for 2 hours, and Eno1 was immunocaptured within the wells of the microplate. The wells of the microplate were washed to remove all other enzymes. Eno1 activity was determined by following the consumption of NADH in an assay buffer that included pyruvate kinase (PK), lactate dehydrogenase (LDH) and the required substrates 2-phospho-D-glycerate (2PG) and NADH. Eno1 converts 2PG to phosphoenolpyruvate, which is converted to pyruvate by PK. Pyruvate is converted to lactate by LDH, and this reaction requires NADH. The consumption of NADH was monitored as decrease of absorbance at 340 nm.

The activity of Enolase-I/G5-dendrimer/SMTP conjugates that were stored at different temperatures at different time points was measured using the assay described above. A concentration of 500 ng of Eno1 was selected for testing because this concentration falls in the middle of the dynamic range of the assay kit. Two different sets of solutions were prepared. One set (control) contained Eno1 alone (i.e. unconjugated Eno1) and the other set contained Eno1/G5-dendrimer/SMTP mixtures. These mixtures were then kept at −80° C., −20° C., 4° C., 22° C., and 37° C. The results showed that in the first week all of the samples were active, and the Eno1/G5-dendrimer/SMTP conjugates seemed to have a slightly higher activity than Eno1 alone. However, the activities of the solutions, regardless of whether or not they contained dendrimers, steadily decreased in the next two weeks. By week 3, the solutions that were stored at 4° C., 22° C., and 37° C. showed no activity, while the solutions that were stored at −80° C., and −20° C. showed significant stability. At the end of the study (Week 10), The Eno1/G5-dendrimer/SMTP solution that was kept at −80° C. retained about 90% of its activity whereas Eno1 alone was only 35% active. On the other hand, Eno1/G5-dendrimer/SMTP solution that was kept at −20° C. was about 24% active, whereas Eno1 alone stored at −20° C. was not active.

### Example 3

### In Vivo Eno1 Targeting Studies with G5 PAMAM Dendrimers

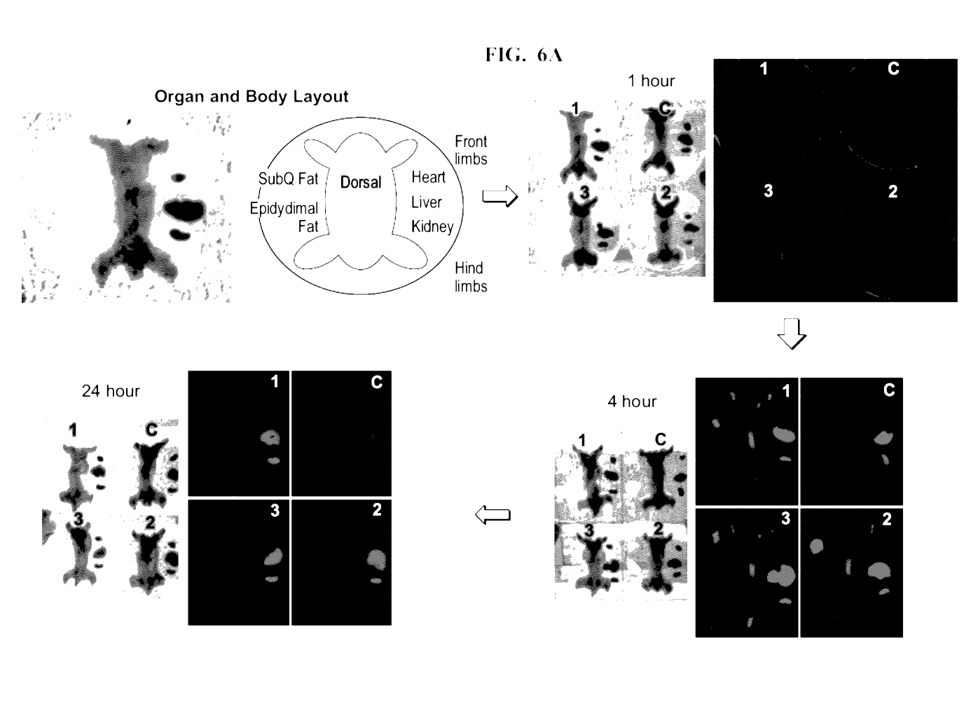

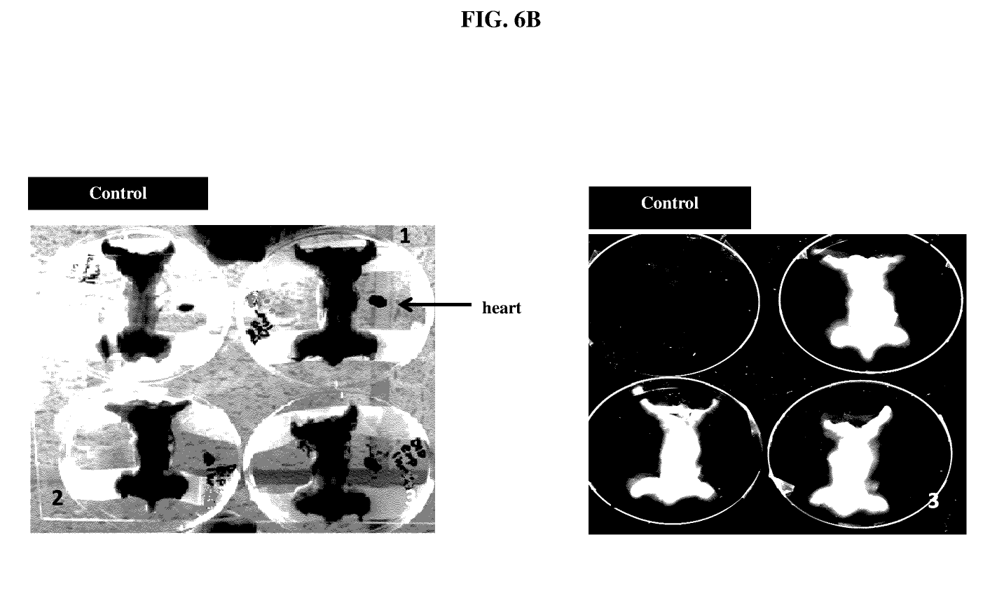

A detectably labeled PAMAM dendrimer complex containing Eno1 was prepared using the method provided in the prior example and analyzed for tissue distribution in mice after subcutaneous injection. Specifically, for 72 hours prior to injection mice were fed alfalfa free food to limit background fluorescence. Mice were injected with 3 μg ENO1/mouse subcutaneously 150 μl total (75 μl left laterally, 75 μl right laterally). The molar ratio of dendrimer to Eno1 in the complex was 5:1. One, 4, and 24 hours post injection animals were sacrificed, skinned, and organs removed in preparation for LI-COR imaging. The results are shown in FIG. 6A.

As shown, at 1 hour, general systemic distribution of the Eno1-PAMAM dendrimer was observed. After 4 hours, significant accumulation of the Eno1-PAMAM dendrimer was observed in liver, kidney, and subcutaneous fat, as well as in the upper torso. After 24 hours, the Eno1-dendrimer complex was substantially cleared and observed substantially in the liver and kidney.

A follow-up study was performed using the skeletal muscle targeted Eno1-PAMAM dendrimer complex containing the SMTP “ASSLNIA”. A detectably labeled PAMAM dendrimer complex containing Eno1 and SMTP ((Enolase-Vivo Tag680xl)-(G5-SMTP)) was prepared using the method provided in the prior example. The molar ratio of dendrimer to SMTP in the complex was 1:1. The experiments were performed essentially as described above. The skeletal muscle targeted Eno1-PAMAM dendrimer complex was administered at a dose of 50 μg/kg body weight. These images in FIG. 6B were taken after 1 hr of injection. Organs, other than the heart, were retained in the body. As can be readily observed, the muscle-targeted Eno1 dendrimer complex was targeted to skeletal muscle, not heart. These results demonstrate that the skeletal muscle targeted Eno1-PAMAM dendrimer complex can be used for the delivery of Eno1 to skeletal muscle cells.

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

### INCORPORATION BY REFERENCE

Each reference, patent, patent application, and GenBank number referred to in the instant application is hereby incorporated by reference as if each reference were noted to be incorporated individually.

### DESCRIPTION OF SEQUENCES

| SEQ | Se- | |
|---|---|---|
| ID NO: | quence | Description |
| 1 | DNA | Human Eno1, transcript variant 1. (FIG. 4B) |
| 2 | AA | Human Eno1, transcript variant 1. (FIG. 4A) |
| 3 | DNA | Human Eno1, transcript variant 2. (FIG. 5B) |
| 4 | AA | Human Eno1, transcript variant 2, also referred to as |
| | | c-myc promoter-binding protein-1 (MBP-1). (FIG. 5A) |
| 5 | AA | muscle targeting peptide (CGHHPVYAC) |
| 6 | AA | muscle targeting peptide (HAIYPRH) |
| 7 | AA | muscle targeting peptide (ASSLNIA) |
| 8 | AA | muscle targeting peptide (WDANGKT) |
| 9 | AA | muscle targeting peptide (GETRAPL) |

We claim:

1. A method of treating obesity in a subject in need thereof, comprising administering to the subject a therapeutically effective amount of a composition comprising Eno1 or a fragment thereof, thereby treating obesity in the subject.

2. The method of claim 1, wherein the subject is suffering from obesity, and wherein the obesity is associated with type 2 diabetes, type 1 diabetes, or pre-diabetes.

3. The method of claim 1, wherein the obesity is caused or exacerbated by a therapeutic treatment.

4. The method of claim 3, wherein the therapeutic treatment is selected from the group consisting of therapeutic agents for the treatment of diabetes, antipsychotic agents, antidepressants, mood stabilizers, anticonvulsants, steroid hormones, prednisone beta-blockers, oral contraceptives, antihistamines, HIV antiretroviral drugs, antiseizure and antimigraine drugs, protease inhibitors, antihyperlipemic agents, hypotensive or antihypertensive agents, anti-obesity agents, diuretics, chemotherapeutic agents, immunotherapeutic agents, and immunosuppressive agents.

5. The method of claim 3, wherein the therapeutic treatment comprises a therapeutic agent for the treatment of diabetes.

6. A method of reducing body weight in a subject afflicted with an overweight condition, comprising administering to the subject a therapeutically effective amount of a composition comprising Eno1 or a fragment thereof, thereby reducing body weight in the subject.

7. The method of claim 6, wherein the subject has a body mass index of between 25 kg/m² and 30 kg/m².

8. The method of claim 6, wherein the overweight condition is caused or exacerbated by a therapeutic treatment.

9. The method of claim 8, wherein the therapeutic treatment is selected from the group consisting of therapeutic agents for the treatment of diabetes, antipsychotic agents, antidepressants, mood stabilizers, anticonvulsants, steroid hormones, prednisone beta-blockers, oral contraceptives, antihistamines, HIV antiretroviral drugs, antiseizure and antimigraine drugs, protease inhibitors, antihyperlipemic agents, hypotensive or antihypertensive agents, anti-obesity agents, diuretics, chemotherapeutic agents, immunotherapeutic agents, and immunosuppressive agents.

10. The method of claim 8, wherein the therapeutic treatment is a therapeutic agent for the treatment of diabetes.

11. A method of reducing or preventing body weight gain in a subject, comprising administering to the subject a therapeutically effective amount of a composition comprising Eno1 or a fragment thereof, thereby reducing or preventing body weight gain in the subject.

12. The method of claim 11, wherein the subject is in need of a therapeutic treatment that induces weight gain.

13. The method of claim 11, wherein the subject is undergoing a therapeutic treatment that induces weight gain.

14. The method of claim 13, wherein the therapeutic treatment is selected from the group consisting of therapeutic agents for the treatment of diabetes, antipsychotic agents, antidepressants, mood stabilizers, anticonvulsants, steroid hormones, prednisone beta-blockers, oral contraceptives, antihistamines, HIV antiretroviral drugs, antiseizure and antimigraine drugs, protease inhibitors, antihyperlipemic agents, hypotensive or antihypertensive agents, anti-obesity agents, diuretics, chemotherapeutic agents, immunotherapeutic agents, and immunosuppressive agents.

15. The method of claim 12, wherein the therapeutic treatment is a therapeutic agent for the treatment of diabetes.

16. The method of claim 5, wherein the therapeutic agent for the treatment of diabetes is selected from the group consisting of sulfonylureas, insulin, GLP-1 receptor agonists, DPP-4 inhibitors, metformin, and rosiglitazone.

17. The method of claim 16, wherein the therapeutic agent for the treatment of diabetes is rosiglitazone.

18-19. (canceled)

20. The method of claim 1, wherein administering the composition comprising Eno1 or the fragment thereof to the subject reduces body weight by at least 5% relative to a control.

21. The method of claim 1, wherein administering the composition comprising Eno1 to the subject reduces body mass index (BMI) by at least 5% relative to a control.

22. The method of claim 1, wherein the subject has any one or more of elevated blood glucose, decreased glucose tolerance, decreased insulin sensitivity and/or insulin resistance, diabetes, elevated Hb1Ac level, and abnormal blood glucose level control.

23. The method of claim 1, further comprising selecting a subject having any one or more of obesity, elevated blood glucose, decreased glucose tolerance, decreased insulin sensitivity and/or insulin resistance, diabetes, elevated Hb1Ac level, and abnormal blood glucose level control.

24. The method of claim 1, wherein the subject is human.

25. The method of claim 1, wherein the Eno1 or fragment thereof comprises an Eno1 polypeptide or a fragment thereof.

26. The method of claim 1, wherein the Eno1 or fragment thereof comprises an Eno1 nucleic acid or a fragment thereof.

27. The method of claim 26, wherein the Eno1 nucleic acid or fragment thereof is present in an expression vector.

28. The method of claim 25, wherein the Eno1 polypeptide or fragment thereof is biologically active.

29. The method of claim 28, wherein the Eno1 polypeptide or fragment thereof has at least 50%, 60%, 70%, 80% or 90% activity of a purified endogenous human Eno1 polypeptide.

30. The method of claim 1, wherein the Eno1 is human Eno1.

31. The method of claim 1, wherein the composition is for delivery to a muscle cell.

32. The method of claim 25, wherein the composition further comprises a muscle targeting moiety.

33. The method of claim 32, wherein the Eno1 polypeptide or fragment thereof and the muscle targeting moiety are present in a complex.

34. The method of claim 32, wherein the muscle targeting moiety is a muscle targeting peptide.

35. The method of claim 33, wherein the complex further comprises a linker.

36. The method of claim 35, wherein the linker is selected from the group consisting of a covalent linker, a non-covalent linkage, and a reversible linker.

37. The method of claim 35, wherein the linker comprises a protease cleavage site.

38. The method of claim 33, wherein the Eno1 is released from the complex upon delivery to a muscle cell.

39. The method of claim 33, wherein the Eno1 and the muscle targeting peptide are present in the complex at a ratio of about 1:1 to about 1:30.

40. The method of claim 1, wherein the composition further comprises a liposome.

41. The method of claim 1, wherein the composition comprising Eno1 or a fragment thereof is administered orally.

42. The method of claim 1, wherein the composition comprising Eno1 or a fragment thereof is administered parenterally.

43. The method of claim 42, wherein the composition comprising Eno1 or a fragment thereof is administered by a route selected from the group consisting of intramuscular, intravenous, and subcutaneous.
